(19) United States
(12) Patent Application Publication
(10) Pub. No.: US 2026/0360521 A1
(43) Pub. Date: Nov. 12, 2026

(54) SYSTEM AND METHOD FOR CONFIGURING RESIDENTIAL CARE ENVIRONMENTS AND AUTHENTICATING ACTIVITIES OF DAILY LIVING

(71) Applicant: Three Strands Consulting LLC, Holland, MI (US)

(72) Inventors: Kurt Dykema, Holland, MI (US); David W Baarman, Fennville, MI (US)

(21) Appl. No.: 19/201,458

(22) Filed: May 7, 2025

(51) Int. Cl. G16H 40/20; G16H 40/67

(52) U.S. Cl. CPC G16H 40/20; G16H 40/67

(57) ABSTRACT

Systems and methods for configuring residential care environments and authenticating Activities of Daily Living (ADLs). Some systems utilize environmental sensors, wearable devices, and mobile computing platforms to establish and validate environments capable of reliable ADL recognition. The system verifies device installation, communication integrity, environmental coverage, and activity detection performance across monitored zones. The system can authenticate care-related activities by correlating multi-source sensor data with registered caregiver and care recipient identifiers. Sensor readings are compared against stored activity profiles to recognize ADL events, which are authenticated and logged with associated time and identity metadata for compliance, care management, or quality improvement. Automated analysis modules may generate personalized care plans, track changes in ADL performance over time, and provide coaching prompts to caregivers based on evolving needs. The system supports family and professional caregiving scenarios and may use privacy-preserving techniques, such as localized video processing, anonymized identity resolution, and structured event representation.

### BACKGROUND OF THE INVENTION

The present invention relates to systems and methods for monitoring and authenticating care-related activities in residential care environments, and more particularly to verifying Activities of Daily Living (ADLs) through sensor-based event recognition, caregiver identification, and residential care environment qualification.

In the current landscape of elderly care, caregivers such as family members and professionals are often overburdened and under-supported. Tracking the quality, quantity, and impact of care services remains largely anecdotal and inconsistently documented. Care recipients and their families often lack reliable means to verify quantitative and qualitative caregiver participation in supporting Activities of Daily Living (ADLs). This lack of transparency can lead to inconsistencies in care, difficulty in assessing caregiver performance, and challenges in coordinating care among multiple caregivers. Additionally, frequent caregiver turnover necessitates ongoing training and monitoring.

Human flourishing and overall well-being are significant considerations in elderly care. Research indicates that interventions designed to support mental health and well-being, such as fostering social engagement, encouraging physical activity, and facilitating personalized care activities, can lead to notable improvements in life satisfaction and mental health. These activities, which often include cognitive stimulation and behavioral exercises, are particularly beneficial in elderly care settings where both physical and mental health are closely intertwined. Additionally, engagement in meaningful activities, such as social interactions, hobbies, or purpose-driven tasks, contributes significantly to overall well-being. By promoting these interventions and regularly monitoring social interaction and physical activity, quality of life can be enhanced, and challenges like social isolation may be reduced.

While the promotion of flourishing through social engagement and active lifestyles is well-documented, integrating these activities into the caregiving environment presents ongoing challenges. Caregivers, whether professional or family members, often struggle to track and adjust their support to effectively enhance the well-being of care recipients. This difficulty is compounded by the lack of consistent and reliable tools to monitor the activities that contribute to the mental and physical health of care recipients, especially in residential care environments.

In elderly care settings, fostering a sense of purpose through daily activities can positively impact both physical health and emotional well-being, leading to improved health outcomes and greater overall happiness. However, integrating purposeful activities into daily caregiving routines remains a challenge, especially in residential care environments where residents may experience social isolation, cognitive decline, or physical limitations.

Existing systems for monitoring Activities of Daily Living (ADLs) often fall short in residential care environments. Many are designed for institutional or clinical use and are not easily adaptable to residential based settings, where conditions and caregiving models vary widely. While some in-home technologies employ motion sensors, wearables, or basic automation tools, these systems lack robust mechanisms for correlating sensor data with specific caregiver and care recipient identifiers, or for verifying caregiver presence in real time. Moreover, current approaches seldom validate the monitoring environment prior to use, and frequently fail to integrate heterogeneous sensor inputs in a manner that enables reliable, auditable records of care activities tied to time, identity, and task classification.

There is a need for a comprehensive system that tracks ADLs accurately and, at the same time, authenticates caregiver involvement. Such a system should work reliably in the home environment, support seamless integration with multiple caregiver roles, and ensure accurate, auditable records of caregiving activities.

### SUMMARY OF THE INVENTION

The present disclosure provides a system and method for configuring residential care environments and authenticating Activities of Daily Living (ADLs) based on multi-source sensor data and identity correlation. The system includes a network of monitoring components, such as wearable devices, environmental sensors, mobile computing platforms, and room-based monitors, that detect, validate, and structure care-related activities associated with registered caregivers and care recipients.

In some embodiments, the system maintains a library of sensor signatures corresponding to common ADLs (e.g., bathing, dressing, mobility assistance), and detects when such signatures are present within a qualified environment. Sensor data from multiple independent sources can be analyzed to recognize ADL events, which can be suitably associated with caregiver and care recipient identifiers to produce authenticated, time-stamped activity records suitable for care verification, reporting, and quality assurance.

In one aspect, a system and method for qualifying a residential environment is provided. The system and method operate by verifying the presence, placement, and operability of room monitors, wearable tracking devices, and environmental sensors across designated living zones. Validation may include per-room testing of detection capabilities, environmental context awareness, and communication stability. The residential environment may be electronically certified for care monitoring upon successful validation and periodically reassessed to maintain qualification.

Automated data analysis modules can be configured to interpret information collected from authenticated ADL events within qualified residential environments. These modules can operate on structured inputs, such as time-stamped activity logs, caregiver-recipient co-location records, health evaluation data, and environmental monitoring output, and may apply rule-based logic, statistical models, or pattern recognition techniques to identify trends, detect deviations, prioritize care interventions, and generate personalized care plan recommendations.

In some embodiments, the system incorporates family-facing interfaces or mobile applications for capturing direct care recipient feedback, such as mood, pain, or engagement levels, and may augment care data streams with passive observational metrics derived from local camera inputs. Privacy-preserving techniques, including local video processing, anonymized facial analytics, and non-identifying event abstraction, may be applied to reduce or eliminate sensitive data exposure.

The current embodiments provide a technical framework for improving elderly care delivery, validating caregiver effort, enhancing care quality measurement, preventing fraud, and supporting caregiver coaching, incentive programs, and cross-entity care coordination through structured data generation and secure system integrations.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. N or should the use of enumeration be construed as excluding from the scope of the invention any additional steps or components that might be combined with or into the enumerated steps or components.

### BRIEF DESCRIPTION OF THE DRAWINGS

FIG. 1 is a schematic diagram illustrating an exemplary system architecture for residential care monitoring and ADL authentication.

FIG. 2 is a schematic diagram illustrating a care evaluation and planning engine configured to analyze structured activity data, health evaluations, and contextual inputs to generate personalized care plans, curated caregiver guidance, and care recommendations.

FIG. 3 is a schematic diagram illustrating a care at home business operating system configured to manage residential care monitoring workflows, authenticated event ingestion, external system integrations, and user-facing care management applications.

FIG. 4 is a block diagram illustrating an example residential floorplan layout for a qualified care monitoring environment, showing the deployment of room-based monitoring devices and registered personal identification devices associated with caregivers and care recipients across defined living zones.

FIG. 5 is a diagram illustrating examples of activities of daily living (ADLs) that may be detected using a combination of room-based monitoring devices and wearable or mobile sensor inputs, including activities such as laundry handling, food preparation, cleaning, dining, mobility transitions, and sleeping. The system correlates multi-modal sensor data to recognize care-related activities while preserving privacy through structured event detection and anonymized observation techniques.

FIG. 6 is a diagram illustrating exemplary data architecture and input correlations to authenticate ADL events, including device co-location, caregiver and care recipient identifiers, temporal proximity, and multiple sensor modalities aggregated for event verification.

FIG. 7 is a schematic diagram of a care plan curation and caregiver coaching system based on structured data analysis and optional pattern recognition modules.

FIG. 8 is a perspective view of a subjective feedback interface implemented on a tablet device, capturing real-time status indicators such as mood, pain, and energy.

FIG. 9 is a process diagram illustrating a facility qualification workflow, including room-by-room validation procedures, baseline ADL testing, and environmental setup verification.

### DETAILED DESCRIPTION OF THE CURRENT EMBODIMENTS

The present disclosure provides a description of exemplary embodiments related to systems and methods for the authentication of Activities of Daily Living (ADLs) and the qualification of residential care environments for ADL monitoring. The described embodiments encompass architectures in which monitoring devices are deployed across multiple physical zones of a residential environment, and wherein activity data from such devices is correlated with identity data from registered caregiver and care recipient devices to authenticate care-related events. The system further supports configuration processes to evaluate the readiness of an environment for ADL monitoring, including verification of sensor coverage, room-based activity detection capabilities, and care recipient-specific ADL profile alignment.

As used herein, the term “residential care environment” refers to any living situation or facility where individuals receive assistance with daily activities, medical care, or support services. This includes, but is not limited to, private homes, assisted living facilities, nursing homes, memory care units, rehabilitation centers, and any other environment designed to provide long-term or short-term care to individuals who require support due to age, medical conditions, disabilities, or other factors. The residential care environment also includes aging-in-place scenarios, where individuals continue to live at home with support provided by periodic visits from professional caregivers, such as nurses, or by family members who assist with daily living activities. The residential care environment can vary in scale and type, from private residences where family members or professional caregivers provide support, to institutional settings like nursing homes or assisted living facilities that offer structured care and medical supervision. This broad definition ensures that the system described herein is adaptable and applicable to various caregiving contexts, supporting both family and professional care scenarios in diverse residential settings.

In some embodiments, the systems and methods described herein may further include additional computational modules configured to operate on authenticated ADL event data collected within qualified environments. These optional modules may support functions such as generating caregiver interaction prompts, delivering training feedback, or synthesizing care plan suggestions based on observed activity patterns. In certain implementations, these capabilities are enabled by structured inputs—such as time-stamped ADL logs, caregiver and care recipient identity correlations, room-specific activity classifications, and longitudinal care metrics—generated by the core authentication and qualification processes. Although these features may enhance the care experience, support coaching workflows, or contribute to higher-level care analytics, they are not required for operation of the core authentication and qualification system disclosed herein.

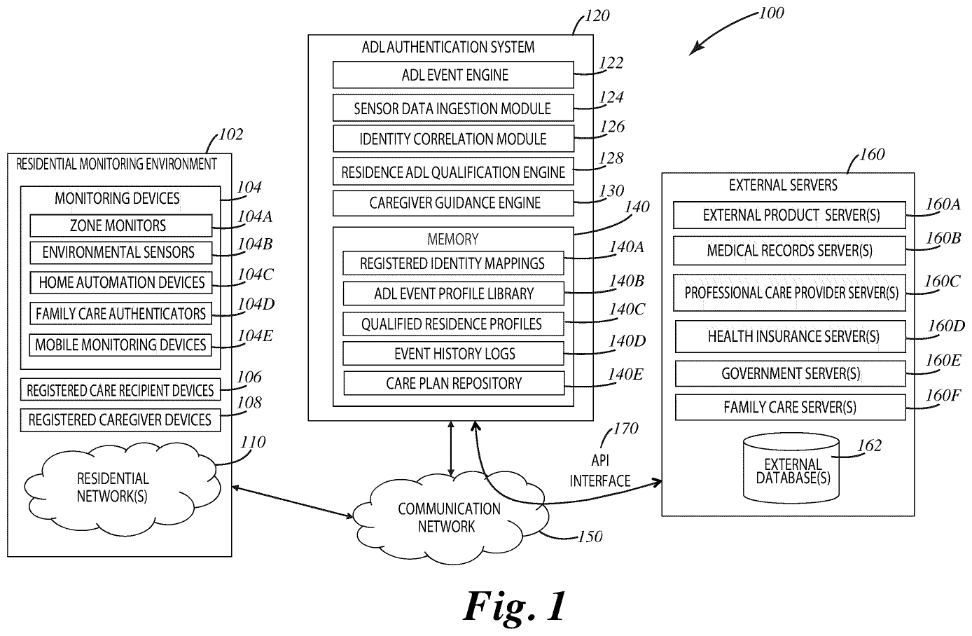

Referring now to FIG. 1, a system architecture 100 is shown, comprising a residential monitoring environment 102, an ADL authentication system 120, a communication network 150, and a plurality of external servers 160. The system is configured to monitor, authenticate, and record Activities of Daily Living (ADLs) within a residential care setting by correlating environmental sensor data with caregiver and care recipient identifiers.

The residential monitoring environment 102 includes a plurality of physical devices configured to detect activities, presence, and contextual information within a residential care setting. These devices include monitoring devices 104, which may be deployed in association with specific locations, carried by individuals, or otherwise configured to generate sensor data relevant to physical activity, environmental conditions, or care-related behavior. Monitoring devices 104 may include zone monitors 104A (e.g., cameras, motion detectors, presence sensors, or microphones configured for activity recognition), environmental sensors 104B (e.g., temperature, humidity, carbon dioxide, or ambient sound level detectors), home automation devices 104C (e.g., smart appliances or ambient assistant devices), family care authenticators 104D (e.g., short-range communication hardware configured to detect caregiver proximity or affirm user presence within a given zone), and mobile monitoring devices 104E, which may include wearable devices, smartphones, or portable computing platforms configured to capture or facilitate capture of motion, biometric, or proximity data while being carried by a caregiver or care recipient.

In addition to the functional classification of monitoring devices, certain devices within the residential monitoring environment may be designated as registered devices—that is, assigned to a specific caregiver or care recipient and associated with a corresponding user identity. These include caregiver devices 108 and care recipient devices 106, which may emit persistent identifiers (e.g., cryptographic tokens, MAC addresses, or Bluetooth beacons) used to correlate sensed activity with a specific individual. In some embodiments, a registered device may additionally perform monitoring functions. For example, a smartwatch worn by a care recipient may operate as both a registered care recipient device 106 and a mobile monitoring device 104E, generating movement data, sensor signals, or proximity confirmations in addition to emitting a registered identifier. This functional overlap enables the system to implement multi-source authentication logic and to accommodate a wide range of hybrid device configurations without imposing rigid distinctions between sensing functionality and identity attribution.

Devices within the residential monitoring environment 102 may communicate over a residential network 110, which may include one or more local wireless communication protocols such as WiFi, Bluetooth Low Energy (BLE), LoRa, Zigbee, or similar technologies. In some embodiments, the residential network 110 may be supported by one or more local hardware components, such as routers, mesh gateways, or home automation hubs, configured to facilitate communication between devices and to relay data toward external systems. Communication with the ADL authentication system 120 may occur either directly—via a communication network 150 such as the Internet or a wide-area wireless network—or indirectly, where data is first routed through the residential network 110 and then passed from a local hardware component to the communication network 150 for delivery to the ADL authentication system 120. This hybrid communication architecture allows for flexible deployment models in which different devices within the monitoring environment may operate independently or in coordination, depending on available connectivity, power constraints, or operational requirements.

In one exemplary embodiment, the ADL authentication system 120 is configured to receive and evaluate data from the residential monitoring environment 102 in order to detect, authenticate, and record Activities of Daily Living (ADLs). The authentication system 120 may be implemented on a cloud-based platform, remote server, or distributed processing node, and may operate continuously or periodically based on available connectivity and system configuration. The system is responsible for identifying ADL-related events by correlating time-stamped sensor data, location metadata, and registered device identifiers with known patterns of care-related activity.

As shown in FIG. 1, the ADL authentication system 120 may include a plurality of functional modules, including an ADL event engine 122, a sensor data ingestion module 124, an identity correlation module 126, a residence ADL qualification engine 128, and a caregiver guidance engine 130. These modules may be implemented in software, firmware, hardware, or any suitable combination thereof, and may be co-located within a single server or distributed across multiple systems.

The system further includes memory 140, which may be used to store a variety of data structures to support system operation. These may include a registered identity mapping store 140A, an ADL event profile library 140B, a qualified residence profile store 140C, event history logs 140D, and a care plan repository 140E, each of which may be implemented using one or more structured or unstructured data stores, such as relational databases, object stores, key-value stores, or document-based systems. In various embodiments, these data structures may be updated dynamically, queried in real time, or synchronized with external systems to support care coordination, compliance tracking, or personalized caregiver feedback.

In some embodiments, one or more of the components of the ADL authentication system 120 may be combined, omitted, or replaced with alternative modules depending on the deployment context, use case, available infrastructure, or desired functionality. The components of the authentication system 120 operate cooperatively to transform low-level activity signals into authenticated care records that may be used for compliance, reporting, quality monitoring, or care plan personalization. In various implementations, the system may support centralized, distributed, or hybrid deployment models, and may accommodate edge-based processing, cloud synchronization, or external system integration as appropriate for the care environment.

In some embodiments, the system may further include a reviewer interface configured to allow authorized reviewers (e.g., family members) to participate actively in the care plan curation process. The family reviewer interface may be accessible through a mobile application, web-based portal, or other authenticated access point integrated with the system infrastructure.

Through the reviewer interface, family members may receive curated care plan recommendations generated by the care evaluation and planning engine. These recommendations may include proposed Activities of Daily Living (ADL) support actions, social engagement opportunities, flourishing interventions, or adjustments to scheduled care routines. The reviewer interface may enable authorized users to accept, defer, prioritize, modify, or decline system-suggested care plan elements based on personal knowledge of the care recipient's preferences, capabilities, or evolving circumstances.

Reviewer decisions may be captured and logged within the system, enabling the care evaluation and planning engine to update the active care plan accordingly. Accepted suggestions may be incorporated into the care recipient's scheduled care routines and caregiver to-do lists, while declined or modified recommendations may trigger further system analysis, personalization adjustments, or feedback loops to refine future care outputs. In some embodiments, the reviewer interface may additionally allow family members to annotate their decisions with optional rationale or contextual information, which may be leveraged by the system to enhance machine learning models or heuristic ranking algorithms.

The system may further track reviewer interaction metrics, such as acceptance rates, declination patterns, reviewer-supplied modifications, or free-form feedback, and may use these inputs to personalize subsequent care recommendations. Reviewer participation may also be factored into longitudinal flourishing trajectory models, enabling a holistic assessment of how active family involvement correlates with care recipient outcomes over time. In some configurations, reviewer-specific access controls, privacy settings, and notification preferences may be configured to align with user permissions, privacy compliance, and care recipient consent parameters.

In some embodiments, user interfaces presented to caregivers, reviewers, or care recipients may be dynamically adapted based on evolving system analysis. Such adaptations may include prioritizing urgent care plan items, adjusting the presentation format of care suggestions, modifying notification timing or intensity, or reordering task lists based on reviewer interaction history, detected care recipient conditions, or predicted engagement patterns. Dynamic user experience adjustments may enhance system responsiveness, support user adherence, and improve overall care plan outcomes.

The ADL event engine 122 is configured to identify and authenticate care-related events that correspond to Activities of Daily Living (ADLs) by evaluating incoming data from the residential monitoring environment 102. The event engine 122 receives input from the sensor data ingestion module 124, including time-stamped activity data from monitoring devices 104, as well as identifiers and activity-related parameters from registered devices 106 and 108. In some embodiments, the ADL event engine 122 accesses one or more ADL event profiles 140B stored in memory 140, each defining a set of conditions or signal patterns that characterize a specific ADL, such as meal preparation, bathing, dressing, or ambulation.

Each event profile 140B may include a predefined combination of required and optional parameters, such as the presence of a caregiver identifier 108 in a qualified zone, detection of motion or object manipulation by a zone monitor 104A, time-of-day constraints, or movement signatures captured by a mobile monitoring device 104E. The event engine 122 applies rule-based or probabilistic logic to compare observed input data against one or more stored profiles. In some embodiments, each profile includes a scoring or confidence model, such that a qualifying event is recognized when the correlation between observed and expected data exceeds a configurable threshold.

Upon successful correlation, the ADL event engine 122 authenticates the event and generates a structured record including metadata such as the event type, timestamp, zone identifier, and correlated caregiver and care recipient identifiers. These authenticated event records may be stored in the event history logs 140D, transmitted to external systems via API interface 170, or passed to other backend components such as the caregiver guidance engine 130 for further processing. In certain implementations, the event engine may also log partial matches, failed authentication attempts, or uncertain events to support auditing, machine learning refinement, or subsequent review.

The sensor data ingestion module 124 is configured to receive, normalize, and prepare incoming data streams originating from devices within the residential monitoring environment 102. These may include raw or pre-processed signals from monitoring devices 104—such as motion detections, environmental measurements, presence confirmations, or image-derived features—as well as identifier and activity-related data transmitted by registered devices 106 and 108. In some embodiments, the ingestion module 124 operates continuously, polling or subscribing to device data feeds via residential network 110 or communication network 150. In other embodiments, it may operate in an event-driven or batch-processing mode, depending on deployment and resource constraints.

Upon receiving device-originated data, the sensor data ingestion module 124 may perform a variety of pre-processing operations, including timestamp normalization, unit conversion, signal filtering, noise suppression, and structural formatting. For example, motion detection signals from multiple zone monitors 104A may be transformed into a standardized event data model with a common timestamp format, location tag, device class identifier, and signal quality score. In some cases, the ingestion module may also extractor infer metadata such as sensor health, signal strength, or device uptime, which may be used to support residence qualification or fault tolerance mechanisms.

The ingestion module 124 passes normalized data payloads to downstream components—such as the ADL event engine 122, identity correlation module 126, and residence ADL qualification engine 128—via one or more inter-process communication mechanisms or shared memory buffers. In some embodiments, the ingestion module may also write raw or partially processed data to temporary buffers or long-term storage, such as a diagnostic trace log or a machine learning training dataset. Optionally, privacy-preserving operations such as data obfuscation, local anonymization, or edge-filtering may be incorporated into the ingestion pipeline to limit the persistence or transmission of sensitive signals. In some embodiments, such operations may occur within the residential monitoring environment 102 itself—on-device or within a local processing hub—such that personally identifiable data is anonymized or reduced before being transmitted over the communication network 150 to the ADL authentication system 120.

The identity correlation module 126 is configured to associate incoming data signals with specific individuals—such as caregivers or care recipients—based on one or more identifiers received from registered devices 106, 108. The correlation module 126 accesses identity mapping records stored in the registered identity mapping store 140A, which may associate each device with a user role, unique identifier, authentication credential, or permission scope. In some embodiments, a registered device may emit one or more identifiers, such as a MAC address, secure token, Bluetooth beacon, or user-specific cryptographic credential, which is received alongside activity data by the sensor data ingestion module 124.

The identity correlation module 126 compares these incoming identifiers to the registered mappings in memory to determine the likely or confirmed identity of the person carrying or operating the device. In some embodiments, the correlation process may involve additional context such as time-of-day, device proximity to specific zones, overlap with zone monitor signals 104A, or detection of expected co-location patterns between caregiver and care recipient devices. These techniques allow the system to correlate identifiers even in the presence of ambiguous or incomplete data, and to support confidence scoring, uncertainty handling, or threshold-based identification logic.

Once a registered identity is confirmed or probabilistically inferred, the identity correlation module 126 appends that information to the corresponding data payload and forwards the enriched data to other system modules, including the ADL event engine 122 and residence ADL qualification engine 128. In some embodiments, identity correlation results may also be stored for audit purposes or used to trigger additional verification prompts in borderline or conflicting scenarios. The module may additionally support revocation, re-registration, or dynamic reassignment of device-user associations to accommodate turnover, temporary assignments, or user device changes.

In some embodiments, the system is further configured to dynamically update the correlation between a device and a user identity based on detected engagement patterns or presence within a monitored zone during an activity period. For example, if a device initially registered to a caregiver exhibits presence within multiple activity zones associated with care delivery (e.g., kitchen assistance, hygiene support) and demonstrates engagement patterns consistent with active caregiving (e.g., interacting with appliances, movement assistance activities), the system may confirm or reinforce the caregiver identity correlation. Conversely, if a device initially unassigned or ambiguously registered exhibits behavioral patterns aligned with care receipt (e.g., stationary presence, passive activity), the system may update the identity correlation to associate the device with a care recipient role. Dynamic identity correlation may occur continuously or at defined intervals during active monitoring periods to enhance system accuracy, support real-time authentication processes, and accommodate changes in device-user association over time.

The residence ADL qualification engine 128 is configured to evaluate whether a given residential care environment is adequately instrumented and properly configured to support the detection and authentication of Activities of Daily Living (ADLs). This evaluation may be performed during an initial setup process, on a periodic basis, or in response to detected anomalies, hardware changes, or system updates. The qualification engine 128 accesses one or more qualified residence profiles 140C stored in memory 140, each of which may define a baseline sensor configuration, acceptable device types, coverage requirements, and zone-specific monitoring capabilities.

In some embodiments, the qualification engine 128 analyzes sensor deployment across multiple rooms or zones within the residence, verifying that each monitored area includes the appropriate types of monitoring devices 104—such as zone monitors 104A, environmental sensors 104B, or family care authenticators 104D—configured to detect the types of activities expected in that space. For example, a bathroom may be expected to include a motion sensor, a sound-level detector, and a family care authenticator capable of verifying caregiver presence. The system may also assess wireless connectivity, signal strength, device uptime, and sensor health metrics received via the sensor data ingestion module 124 to confirm real-time operational readiness.

In some embodiments, the system may also be configured to operate within minimally instrumented residential care environments where only a partial subset of expected monitoring devices is available. In such configurations, adaptive authentication techniques may be employed, including the use of reduced confidence thresholds, simplified event inference models, or proximity-only correlation strategies. These fallback techniques enable continued detection and authentication of care-related activities even in environments where full sensor deployment is impractical, incomplete, or degraded over time.

In some embodiments, privacy-preserving features, such as on-device event recognition, local anonymization of audio/visual data, or encrypted metadata transmission, may be selectively activated, disabled, or dynamically configured to comply with jurisdiction-specific privacy regulations or user-configured confidentiality settings.

The qualification engine 128 may score each zone or the residence as a whole against one or more ADL qualification templates or thresholds. In some embodiments, the system may generate a pass/fail result, a qualification level (e.g., full, partial, insufficient), or a recommendation to add or reposition specific devices. Residences that fail qualification may be flagged within the system, limiting or suppressing downstream authentication processing for that environment until qualification requirements are met. In some implementations, qualification results may be logged, reviewed, or transmitted to external systems to support care compliance reporting, facility readiness audits, or sensor deployment planning.

In some embodiments, the qualification engine may further categorize the baseline ADL profile into ADL types, such as hygiene-related activities, nutrition-related activities, and mobility-related activities. The qualification engine may evaluate room readiness separately for each ADL type, such that the ability of each monitored zone to detect hygiene-related ADLs, nutrition-related ADLs, and mobility-related ADLs can be independently confirmed. In this manner, room qualification can be refined to reflect the specific types of activities supported within each zone, thereby improving system accuracy, personalized care planning, and the reliability of ADL event authentication.

The caregiver guidance engine 130 is an optional module configured to generate prompts, recommendations, or personalized feedback for caregivers based on authenticated ADL events, behavioral patterns, and care context. The engine may operate in real time, in response to system-detected events, or as part of a scheduled care review cycle. The caregiver guidance engine 130 accesses content from a care plan repository 140E, which may include predefined guidance templates, curated care routines, coaching messages, or activity-based reminders.

In some embodiments, the guidance engine 130 monitors authenticated event records generated by the ADL event engine 122 and stored in the event history logs 140D. Based on activity frequency, caregiver involvement, missed ADLs, or caregiver-recipient interaction metrics, the engine may determine when and how to deliver actionable guidance. This may include short-term prompts (e.g., reminders to assist with a missed meal), long-term coaching suggestions (e.g., encouragement to diversify ADL engagement), or behavioral trend summaries delivered via a mobile app, dashboard, or third-party care platform.

In some embodiments, the qualification engine may apply a weighted scoring algorithm to evaluate environment readiness. Individual ADL types, such as hygiene-related activities, nutrition-related activities, and mobility-related activities, may be assigned detection value scores reflecting their relative importance based on care recipient-specific configuration data or care plan ranking. For example, hygiene-related activities may be assigned greater weight in the environment qualification process if a care recipient's care plan emphasizes hygiene support. The weighted scoring algorithm may aggregate detection scores across monitored zones to determine whether the overall residential care environment satisfies a coverage threshold for ADL authentication.

The caregiver guidance engine 130 may also consider non-activity signals, such as sleep patterns, device usage history, or caregiver fatigue indicators, where available. In some embodiments, guidance outputs may be triggered based on configurable thresholds or rule sets and may be filtered according to user role, device permissions, or privacy preferences. For example, family caregivers may receive supportive prompts, while professional caregivers receive compliance-relevant recommendations. The engine may also generate care plan updates or escalate unresolved care concerns to external systems or supervisory users.

In some embodiments, the caregiver guidance engine 130 or associated backend infrastructure may be configured to support a caregiver incentive framework, wherein authenticated ADL activities, care quality metrics, or engagement milestones are associated with microbonuses, recognition awards, or tiered achievement statuses. Incentive structures may be dynamically generated based on care plan adherence, flourishing score improvements, task completion rates, or caregiver interaction quality metrics. Microbonuses may include monetary rewards, virtual achievements, eligibility for professional development opportunities, or other tangible and intangible benefits designed to encourage sustained caregiver engagement and quality care delivery. In some implementations, incentive metrics and reward notifications may be surfaced through caregiver mobile applications or administrative dashboards integrated within the system.

In some embodiments, the ADL authentication system 120 may be configured to exchange data with one or more external servers 160 via an API interface 170. These external servers may include third-party systems, institutional platforms, or caregiver-accessible tools that provide supplemental context, consume authenticated ADL records, or support system interoperability. Communications via API interface 170 may occur over the communication network 150 and may involve structured data formats such as JSON, XML, HL7, or proprietary protocol formats. In various embodiments, API interactions may be uni- or bi-directional and may include query, push, subscription, or event-driven exchange models. Data exchanged via API interface 170 may include user metadata, system configuration details, care plan content, audit logs, reimbursement requests, or ADL verification records.

External product servers 160A may include device-specific or platform-specific cloud services associated with monitoring devices 104 or registered devices 106, 108. For example, a wearable manufacturer may expose real-time heart rate, step count, or location history via an authenticated API, enabling the ADL authentication system 120 to ingest enriched sensor signals without relying on local device polling. In some embodiments, external product servers may also provide firmware updates, device health information, or error reporting that feeds into residence qualification logic or system diagnostics. Integration with such servers may also enable passive sensor validation or retrospective data access for ADL event verification.

Medical records servers 160B may include electronic health record (EHR) platforms, personal health information systems, or payer-provider care coordination databases. Through API interface 170, the system may retrieve relevant health context—such as diagnoses, allergies, mobility restrictions, or medication regimens—that can inform event profile matching, caregiver prompting logic, or care plan personalization. In some embodiments, authenticated ADL event records may also be exported to the medical records server to support clinical decision-making, discharge planning, or cross-provider coordination.

Professional care provider servers 160C may include systems operated by home health agencies, care coordinators, skilled nursing facilities, or other institutional care providers. These systems may interface with the ADL authentication system 120 via API interface 170 to facilitate workforce coordination, service verification, or care quality monitoring. For example, a provider's scheduling system may submit caregiver assignment data to associate care activities with specific personnel, or may receive authenticated ADL records as part of visit documentation. In some embodiments, provider servers may also use ADL event data to trigger alerts, update task checklists, or inform care team dashboards. Bidirectional integration may support shift-based credentialing, fraud detection, or comparative performance analysis across multiple caregivers or facilities.

Health insurance servers 160D may include systems maintained by private insurers, Medicare/Medicaid administrators, or long-term care benefit managers. Through the API interface 170, the ADL authentication system 120 may submit event verification records as part of claims processing, eligibility determination, or service authorization workflows. For example, a verified record of assisted bathing or mobility support may be used to document compliance with a care plan and trigger reimbursement or coverage continuation. In some embodiments, the system may also receive payer-side rule updates, utilization thresholds, or audit triggers to ensure compliance with plan-specific documentation requirements. Data sharing may be governed by healthcare interoperability standards and may support automated submission pipelines, appeals handling, or payer-specific outcome metrics.

Government servers 160E may include systems operated by regulatory bodies, elder care agencies, or public health departments. The ADL authentication system 120 may transmit selected event summaries, compliance records, or de-identified population-level statistics to such servers via the API interface 170. In some embodiments, authenticated ADL records may serve as digital evidence to support qualification for care stipends, tax credits, or dependent care benefits under government programs. Government integration may also enable cross-validation of caregiver licensure, background checks, or care recipient eligibility status. Additionally, policy changes, reporting obligations, or program-specific criteria may be retrieved from government servers to ensure alignment with public standards and accountability frameworks.

Family care servers 160F may include applications or portals designed to allow authorized family members or informal caregivers to receive updates, provide input, or access historical ADL data. Through the API interface 170, the ADL authentication system 120 may push activity summaries, behavioral trends, or care plan adherence metrics to a family-facing system for transparency and engagement. In some embodiments, family members may configure notification preferences, contribute qualitative observations, or respond to feedback requests within a shared care interface. These integrations may help facilitate hybrid care models where professional and family caregivers operate collaboratively, and may provide continuity of care during transitions, vacations, or staffing changes.

In some embodiments, the system may further facilitate peer support networks for family caregivers through integration with family care servers 160F or associated mobile applications. Authorized family caregivers may participate in moderated peer groups, resource-sharing forums, or virtual support communities to exchange advice, share caregiving experiences, or access curated educational materials. The peer support system may offer topic-specific discussion channels, structured mentoring programs, and event-driven group activities aligned with common caregiving challenges or flourishing score improvement goals. Participation metrics, resource utilization, and engagement outcomes may be optionally integrated into caregiver guidance workflows or flourishing trajectory modeling to enhance overall care ecosystem resilience and support family caregiver well-being.

In some embodiments, one or more external databases 162 may be accessible to or integrated with external servers 160A-160F. These databases may store supplemental user data, medical histories, care plans, credentialing records, or historical event logs maintained by third-party platforms. The ADL authentication system 120 may interact with such databases indirectly—via corresponding external servers—or directly, where permitted, through the API interface 170. For example, a family care server 160F may access a shared document database containing historical care summaries, while a government server 160E may maintain a secure data store of program eligibility criteria or audit flags. External databases may use relational, document-based, or object-based storage architectures and may support data replication, synchronization, or federated query access depending on the deployment context.

Although the different elements and assemblies of the embodiments are described herein as having certain functional characteristics, each element and/or its relation to other elements can be depicted or oriented in a variety of different aesthetic configurations, which support the ornamental and aesthetic aspects of the same. Simply because an apparatus, element or assembly of one or more of elements is described herein as having a function does not mean its orientation, layout or configuration is not purely aesthetic and ornamental in nature.

Integration with external servers 160 and external databases 162 is optional and may be selectively enabled, disabled, or customized based on deployment configuration, privacy requirements, or care environment preferences. In some embodiments, the ADL authentication system 120 may operate entirely within the residential monitoring environment 102 and backend system infrastructure without external system communication. In other embodiments, selective integration with one or more external servers may be used to enrich ADL authentication logic, supplement caregiver guidance, enable compliance reporting, or facilitate care plan personalization. API interface 170 may be configured to enforce data access controls, filter outbound information, or authenticate inbound requests to maintain system security and user privacy across external interactions.

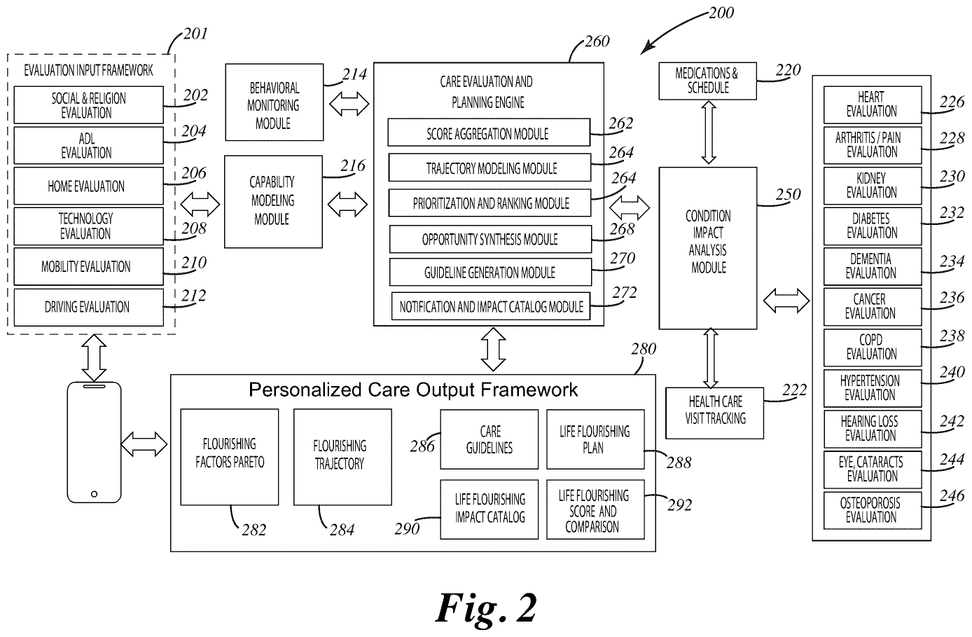

In some embodiments, authenticated ADL records and associated care interaction data generated by the ADL authentication system 120 may be further processed by a separate, higher-level analytics module configured to generate personalized care plans, evaluations, and caregiver guidance. As shown in FIG. 2, this analytics module-referred to herein as the care evaluation and planning engine 260, part of the care optimization and planning system 200 may consume structured data from a variety of sources, including the event history logs 140D, caregiver identity data from memory 140A, and additional external inputs such as medical evaluations, health record servers 160B, and user-supplied contextual information. The system of FIG. 2 is modular and may be optionally deployed as an extension to the core authentication platform, enabling enhanced functionality without altering the baseline ADL verification framework.

The care evaluation and planning engine serves as an analytic module, responsible for curating personalized care plans and providing actionable insights for both caregivers and care recipients. It utilizes a variety of data inputs, including ADL event records, caregiver and care recipient identities, medical evaluations, and additional external data sources. The engine dynamically updates care plans based on evolving data and the changing needs of the care recipient.

The engine's functionality can be further enhanced by incorporating insights from social and family data, health records, and environmental sensors. In certain embodiments, machine learning algorithms are used as part of the engine to assist in identifying care recipients who may need urgent intervention. This analysis prioritizes individuals based on a holistic view of factors, such as family history, medical assessments, wearable data, and environmental conditions. This integration ensures that the system can adapt and respond to the evolving needs of care recipients in a dynamic, data-driven manner, supporting the ongoing refinement of personalized care plans.

Leveraging the analysis of multi-source data, the care planning engine (augmented by machine learning capabilities) can identify care recipients at risk by cross-referencing multiple data domains (e.g., deteriorating health, social isolation, poor mobility, and declining sleep quality). The system can automatically generate detailed profiles for each care recipient, summarizing key risk factors such as chronic conditions, mobility issues, social support gaps, or sleep disturbances. For each identified care recipient, the engine recommends personalized, actionable recommendations that take into account the individual's unique medical needs, lifestyle preferences, and existing support systems.

These personalized recommendations may include, for example, dietary adjustments tailored to the individual's medical condition and cultural preferences, exercise plans based on mobility limitations, or social engagement activities involving family members or caregivers. The system ensures that these recommendations are not only realistic and actionable but also socially integrated, aiming to improve adherence by incorporating the care recipient's social network into the care process.

In addition to health-related recommendations, the engine can integrate insights from social data, including family surveys and wearable data. The system can assess the risk of social isolation by analyzing wearable data—such as heart rate variability and step counts—which may signal signs of depression or stress. Upon detecting isolation risks, the engine generates personalized recommendations for social engagement activities, tailored to the care recipient's interests, family connections, and support network. These activities are designed to foster meaningful interactions, thus helping reduce feelings of loneliness and enhance overall well-being.

For example, a care recipient with limited mobility might be encouraged to engage in a virtual game with family members or to participate in a shared hobby with a caregiver. These activities are carefully designed to align with the care recipient's preferences and social support systems, ensuring that the engagement remains both relevant and sustainable.

The engine is also capable of providing continuous monitoring and feedback, ensuring that care plans remain effective and responsive over time. It analyzes real-time data from the residential environment, including wearables, environmental sensors, and caregiver inputs. When deviations from expected patterns are detected, such as reduced activity levels, changes in mobility, or altered social interaction, the system triggers automatic adjustments to the care plan. This ensures that interventions are timely, appropriate, and aligned with the care recipient's evolving needs.

Additionally, the system generates feedback for caregivers, offering them actionable insights based on authenticated ADL events. This feedback may include reminders to address missed activities or summaries of behavioral trends that help caregivers stay informed and proactive in their care delivery.

As the system accumulates more data over time, incorporating feedback from caregivers and monitoring changes in care recipient behavior, the care plan can be dynamically refined. The system continually cross-references various data points, including medical history, social connections, and activity patterns, to update care recommendations. This evolving process ensures that care plans are responsive to the care recipient's changing needs and remain focused on promoting long-term well-being.

The care evaluation and planning engine may integrate inputs from multiple domains—such as health conditions, ADL engagement history, home environment evaluations, mobility status, and caregiver interactions—to produce summary scores, care guidelines, prioritized care opportunities, and impact evaluations. These outputs may be used to support care personalization, quality improvement, or patient/caregiver engagement. The resulting recommendations or scores may be transmitted to family care servers 160F, professional care provider servers 160C, or rendered locally on a mobile application or dashboard. The architecture of FIG. 2 illustrates an example embodiment in which multiple data streams converge within an analytics platform to drive actionable care insights centered on user flourishing and system coordination.

As shown in FIG. 2, the care evaluation and planning engine receives inputs from a range of assessment domains, collectively referred to as the evaluation input framework 201. These include, without limitation:
Social and Religion Evaluation 202
ADL Evaluation 204
Home Evaluation 206
Technology Evaluation 208
Mobility Evaluation 210
Driving Evaluation 212

Each of these inputs may represent structured evaluation data gathered through formal assessments, observation, or system interaction. These evaluations are collectively processed to produce a synthesized view of the individual's care context.

The care evaluation and planning engine includes a capability modeling module 216 configured to generate a personalized representation of the user's functional and engagement profile. This profile is constructed based on structured evaluation data from the evaluation input framework 201, including assessments such as ADL performance 204, mobility 210, technology familiarity 208, and other functional or cognitive evaluations 202-212.

The system may synthesize these inputs to produce domain-specific capability indicators-such as prompting responsiveness, physical independence, digital readiness, or environmental adaptability. These indicators may be assigned scores, categories, or thresholds and may be used to define the scope of feasible care interventions. The capability modeling module 216 operates as an intermediate analysis layer that distills complex, multi-domain assessments into a structured capability profile, which is then passed to the care evaluation and planning engine for personalized care synthesis.

The behavioral monitoring module 214 may also inform time-sensitive care planning, including identifying ideal times for prompting, escalating care activities, or adjusting scheduled evaluations. In some embodiments, behavioral monitoring data may be linked to adaptive learning modules that refine system prompts or scheduling recommendations based on user habits and observed preferences. Although not a standalone evaluation, the behavioral insights captured through 214 help contextualize and refine the synthesis performed by the core engine and inform downstream recommendation generation.

The care evaluation and planning engine may incorporate medical, clinical, and contextual health data received from a plurality of domain-specific evaluation modules and medication tracking systems. These inputs are processed through a condition impact analysis module 250, which synthesizes disease-related evaluations, treatment constraints, and comorbidity considerations into structured insights that can be used to personalize care plans and opportunity prioritization.

As shown in FIG. 2, the condition impact analysis module 250 may receive input from one or more disease-specific evaluation modules, including but not limited to: heart evaluation 226, arthritis and pain evaluation 228, kidney evaluation 230, diabetes evaluation 232, dementia evaluation 234, cancer evaluation 236, chronic obstructive pulmonary disease (COPD) evaluation 238, hypertension evaluation 240, hearing loss evaluation 242, eye or cataracts evaluation 244, and osteoporosis evaluation 246. Each of these evaluations may be structured or semi-structured and may be derived from external systems such as medical records servers 160B or manually input by clinicians or caregivers.

In addition to disease-specific inputs, the module may receive medications and schedule data 220, including drug names, dosages, side effect profiles, and adherence metrics, as well as healthcare visit tracking data 222, which may include visit frequency, missed appointments, or episodic intervention history. The module may perform logic to identify comorbid interactions, score individual conditions, and generate a composite health impact summary. It may further identify the most impactful conditions—such as the top three or top ten based on severity, frequency, or complexity—and quantify limitations imposed by medication regimens or treatment side effects.

This synthesized clinical insight is then passed into the care evaluation and planning engine to inform the interpretation of ADL capability scores, customize care guideline recommendations, and prioritize or suppress certain opportunity pathways based on medical constraints.

In some embodiments, the care evaluation and planning engine may be accessible through a mobile device interface, such as a smartphone or tablet application. As illustrated in the lower left corner of FIG. 2, a mobile application may serve as both a user input portal and a results delivery interface, allowing caregivers, care recipients, or authorized family members to interact with the system. Through this interface, users may provide contextual information, respond to care prompts, or configure preferences related to scheduling, activity monitoring, or intervention sensitivity.

The interface may also display system-generated outputs—such as care plan summaries, capability indicators, personalized recommendations, or comparative scores—derived from the analytics performed within the care evaluation and planning engine. In some embodiments, the interface may enable secure bidirectional communication with external servers 160F or 160C, facilitating hybrid care coordination across professional and informal care networks. The use of a mobile application may support enhanced engagement, transparency, and user-directed configuration of the system's care optimization features.

In some embodiments, the care evaluation and planning engine 260 includes a plurality of internal submodules configured to analyze synthesized user data, rank identified care priorities, and generate personalized outputs for caregiver or system-directed intervention. These submodules may operate independently or in coordination to transform multi-domain assessment data, clinical context, and behavioral observations into structured, actionable care artifacts.

The engine may include a score aggregation module 262, which is configured to compute one or more composite scores reflecting the overall care status or life flourishing potential of the user. This module may produce a global Life Flourishing Score, as well as domain-specific indicators derived from evaluation inputs 201, capability modeling outputs 216, and clinical synthesis data from the condition impact analysis module 250. Scores may incorporate dynamic weightings that adjust based on temporal patterns or care context.

A trajectory modeling module 264 may be provided to estimate care-related trends over time. This module may construct historical engagement curves, detect behavioral decay or improvement patterns, or project forward-looking trajectories indicating expected progression in ADL function, caregiver dependency, or intervention response. Trajectories may be used to time interventions, identify periods of increased risk, or assess the potential efficacy of proposed care changes.

The system may further include a prioritization and ranking module 266, configured to generate structured representations of user limitations, care needs, system-detected concerns, and high-impact contributors to care complexity. In some embodiments, the module may apply Pareto-style logic—i.e., identifying a subset of dominant issues accounting for a majority of impact—or other heuristic ranking techniques to produce ordered lists. Ranked profiles may include limitation prioritization outputs, needs-based rankings, and care bottleneck indicators.

An opportunity synthesis module 268 may generate a curated list of care opportunities, improvement targets, or behavioral interventions based on the user's capability model and ranked concern profiles. This module may suppress suggestions that exceed the user's functional capacity or conflict with medical constraints identified via module 250. In some embodiments, opportunity synthesis may also reflect caregiver burden indicators or user preferences.

The engine may include a guideline generation module 270, configured to produce personalized care recommendations, prompts, or structured plans. These guidelines may incorporate predefined clinical rules, user-specific preferences, or institutional policy frameworks. Generated guidelines may be static (e.g., standing daily hygiene reminders) or dynamic (e.g., modified prompting following a missed meal ADL event).

Finally, a notification and impact catalog module 272 may format, package, or deliver outputs to external interfaces, such as mobile applications or caregiver dashboards. This module may curate and tag system outputs such as the Life Flourishing Score, summary recommendations, care trajectory plots, or engagement alerts for prioritized delivery. In some implementations, module 272 may also handle adaptive timing, escalation logic, or output suppression based on user or system context.

The personalized care output framework 280 includes a set of structured outputs generated by the care evaluation and planning engine 260. These outputs are derived from integrated analysis across multiple domains, including user capabilities, behavioral patterns, evaluation results, and medical constraints. As shown in FIG. 2, the output framework 280 may include six principal system-generated documents and recommendations, each serving a distinct role in care guidance, user engagement, and performance tracking.

A flourishing factors Pareto 282 represents a ranked distribution of care-related factors, organized to highlight the relative impact of various needs, limitations, or behavioral contributors to the user's flourishing potential. This output may be generated by the prioritization and ranking module 266 and may present a visualization or structured report that identifies which factors most significantly influence the user's well-being. In some embodiments, the flourishing factors Pareto 282 may be used to guide caregiver focus or inform high-level planning discussions.

A flourishing trajectory 284 illustrates the user's historical or projected change in care status over time. This output may be generated by the trajectory modeling module 264 and may reflect ADL performance patterns, response to care interventions, or modeled forecasts of decline, improvement, or stability across monitored domains. The trajectory may be rendered visually, numerically, or descriptively, and may include annotated inflection points—such as “decline in hygiene engagement,” “increased caregiver prompting required,” or “stabilization of mobility score”—to support care planning decisions and caregiver coordination.

The system may generate care guidelines 286, representing structured instructions, prompts, or recommendations aligned with the user's prioritized needs, capability profile, and clinical context. These guidelines may be derived from rule-based logic within the guideline generation module 270 and may address domains such as hygiene routines, physical activity goals, cognitive stimulation strategies, or caregiver engagement recommendations.

A life flourishing plan 288 may be assembled to integrate prioritized opportunities, suggested interventions, and contextual recommendations into a coherent care narrative or action strategy. The plan may be curated by the opportunity synthesis module 268 and formatted for delivery to caregivers, family members, or case managers. The life flourishing plan 288 may include targeted goals, task lists, or projected benefits tied to system-detected improvement areas.

A life flourishing impact catalog 290 may be generated to present the anticipated or actual effects of implemented care actions, changes in user status, or systemic interventions. This catalog may include descriptive entries, metrics, or event-based annotations and may be maintained over time to support feedback loops or longitudinal outcome tracking. The catalog may be populated through contributions from multiple submodules, including 264 (trajectories), 266 (rankings), and 272 (notifications and impact reporting).

Finally, the system may output a life flourishing score and comparison 292, which reflects a current or composite score derived from the score aggregation module 262 and benchmarked against prior scores, peer groups, or care objectives. This score may be used to evaluate intervention efficacy, track progress toward predefined goals, or inform payer-facing outcome documentation.

Each output in the framework 280 may be transmitted via system interface, rendered on a user-facing display, or exported to external servers for integration into third-party care systems.

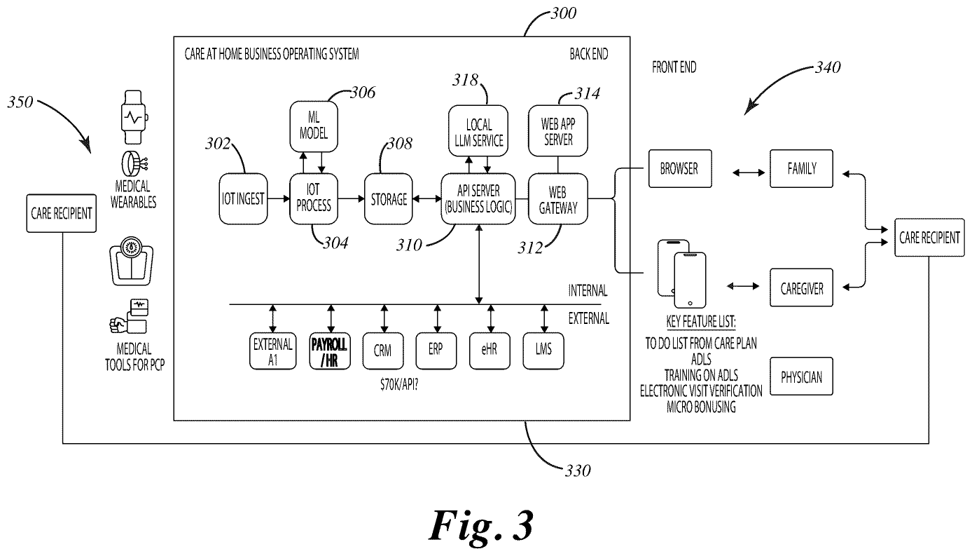

In some embodiments, the residential monitoring environment 102, the ADL authentication system 120, and the care evaluation and planning engine 260 may be integrated with or interface to a broader business operations coordination system, such as the exemplary system illustrated in FIG. 3. In such embodiments, certain modules of the business operating system, such as the IoT ingest module, the IoT processing module, and the storage infrastructure may be configured to receive, normalize, or persist data originating from the residential monitoring and ADL authentication systems. For example, sensor data captured and processed by the sensor data ingestion module 124 or authenticated ADL event records generated by the ADL event engine 122 may be transmitted to the IoT ingest module for aggregation and further processing.

The IoT processing and storage modules may similarly handle care-related information, such as authenticated ADL events, caregiver interaction records, and flourishing scores, alongside non-care-specific operational data originating from external devices, wearables, or administrative systems. In other embodiments, the business operations coordination system may operate independently of the ADL authentication and care evaluation systems, receiving and managing device data streams, business logic workflows, and operational records unrelated to residential monitoring. This modular architecture allows for flexible deployment models in which care authentication, care planning, and business management systems may operate cooperatively or independently, depending on the needs of the deployment environment.

Referring now to FIG. 3, an exemplary embodiment of a care business operating system 300 is illustrated. The care business operating system 300 is configured to coordinate, process, and manage a variety of care-related, administrative, and operational data streams. The system 300 may operate in conjunction with the residential monitoring environment 102, the ADL authentication system 120, and the care evaluation and planning engine 260 described above, or may operate independently in configurations where broader business coordination functionality is desired. System 300 includes a plurality of internal modules configured to receive data from a variety of sources, process and store relevant information, interact with external systems and services 330, and deliver user-facing outputs through a front-end access layer 340.

As shown in FIG. 3, the care business operating system 300 may receive input data from a variety of sources, collectively referred to herein as the resident and medical data sources 350. These sources may include devices or systems situated within a residential care environment or associated with external clinical care providers. Resident and medical data sources 350 may include, for example, direct sensor inputs associated with the resident, such as activity trackers, presence sensors, or ambient monitoring systems. Sources may also include medical wearable devices, such as smartwatches, heart rate monitors, glucose monitors, or fall detection devices configured to capture physiological or mobility-related data.

In some embodiments, resident and medical data sources 350 may further encompass medical tools associated with primary care providers, such as stethoscopes, blood pressure monitors, pulse oximeters, or other clinical evaluation devices capable of transmitting data to the backend system 300. Data streams from resident and medical data sources 350 may be transmitted directly to an IoT ingest module 302 for initial reception and normalization, as described in further detail below. The flexible architecture of system 300 allows it to accommodate diverse input types and evolving medical technologies without requiring redesign of core system components.

Data received from the resident and medical data sources 350 may initially be processed by an IoT ingest module 302 within the care business operating system 300. The IoT ingest module 302 can be configured to receive heterogeneous data streams from a variety of connected devices, normalize incoming data formats, validate transmission integrity, and apply preliminary filtering or categorization based on device type, data origin, or content characteristics. In some embodiments, the IoT ingest module 302 may also perform initial time-stamping, zone-mapping, or device authentication procedures to ensure reliable downstream processing.

Normalized data may then be passed to an IoT processing module 304, which is configured to perform intermediate-level data handling tasks, including context enrichment, aggregation of related data points, event reconstruction, or preliminary feature extraction. The IoT processing module 304 may, for example, aggregate multiple motion sensor activations to form a composite ADL event estimate or combine physiological readings to generate a multi-parameter health snapshot.

Processed data may optionally be analyzed by a machine learning analysis module 306, which may apply predictive models, anomaly detection algorithms, or classification engines to identify patterns or deviations relevant to care planning, caregiver prompting, or operational decision-making. In some embodiments, the machine learning analysis module 306 may operate locally, while in other embodiments it may interface with external analytics services via integration pathways, such as those described herein.

Both raw and processed data may be selectively stored within a storage infrastructure 308. The storage infrastructure 308 may comprise one or more structured or unstructured data stores configured to persist event logs, biometric measurements, ADL engagement records, caregiver interactions, or business operations data. Stored data may be indexed, encrypted, and access-controlled according to deployment-specific security and compliance policies, enabling subsequent querying, reporting, or integration with external systems.

The care business operating system 300 may include an API server 310 configured to manage structured internal communications between system modules and user-facing services. The API server 310 may expose standardized interfaces for accessing system resources, including RESTful APIs, GraphQL endpoints, or other communication protocols, and may facilitate authenticated data exchange among backend processing modules and the front-end access layer 340. The API server 310 may implement request validation, permission checking, and session management to support robust internal interoperability.

Data or service requests processed through the API server 310 may be routed to a web gateway 312, which functions as an access control and traffic management layer for web-based and mobile-based client interactions. The web gateway 312 may manage secure session handling, encryption/decryption tasks, load balancing, firewalling, and throttling of user or system interactions. In some embodiments, the web gateway 312 may apply role-based access control rules to differentiate between family member access, caregiver access, and clinical care provider access based on authentication credentials.

Client-facing services, including user dashboards, task management portals, and care plan interfaces, may be hosted on a web application server 314. The web application server 314 may render personalized views, dynamic task lists, electronic visit verification workflows, micro-bonusing feedback modules, and training content associated with ADL procedures. User interfaces rendered by the web application server 314 may be accessible via browser-based platforms or dedicated mobile applications forming part of the front-end access layer 340, as described in further detail below.

In some embodiments, the care business operating system 300 may include a local language model module 318 configured to support intelligent processing of structured prompts, care planning queries, summarization tasks, or other natural language processing (NLP) operations. The local language model module 318 may be deployed within the backend infrastructure to enable privacy-preserving, low-latency processing of user or system-generated prompts without transmission of sensitive data to external services. In some embodiments, the local language model module 318 may leverage fine-tuned natural language models, generative models, or custom heuristic engines to assist with automated documentation, caregiver guidance generation, or activity summarization. In other embodiments, or when additional computational resources are desired, the system 300 may selectively route structured queries to external LLM services, such as those accessed through the external systems and services interfaces 330, described below.

In addition to managing internal communications, the API server 310 may be configured to route structured communications to external systems and services 330. Through secured APIs or service buses, the API server 310 may enable modular interaction with external enterprise systems, external analytics providers, or third-party service platforms, as further described below. The API server 310 may handle authentication, protocol translation, and message formatting to support reliable and secure communication with external entities.

As shown in FIG. 3, the external systems and services interfaces 330 represent a grouping of external enterprise platforms and service providers accessible via the API server 310. These external systems 330 may include customer relationship management (CRM) systems, enterprise resource planning (ERP) systems, electronic health record (eHR) systems, learning management systems (LMS), and external artificial intelligence model services, such as external large language models (LLMs). Structured communication with these external systems may be facilitated through secured APIs, event-driven webhooks, or scheduled synchronization tasks, enabling the care business operating system 300 to extend its functionality into broader administrative, clinical, and operational ecosystems.

In some embodiments, external systems integration through 330 may enable synchronization of user profiles, care plan metadata, compliance documentation, training certifications, financial records, or visit verification logs between the care business operating system and organizational back-office systems. Data interchange may occur via secured API calls, scheduled batch operations, or event-driven push notifications, depending on the configuration of external systems.

Additionally, the external systems and services interfaces 330 may optionally enable selective access to external AI model services, including large language model (LLM) providers. In configurations where additional natural language processing resources are required, the system 300 may route structured prompts or analytics requests to external LLM services accessible via the external integration interface, thereby enabling hybrid AI-assisted workflows without requiring exclusive reliance on internal LLM module 318 resources. This flexible architecture allows the system to balance privacy, computational efficiency, and functional extensibility based on deployment-specific requirements.

The care business operating system 300 may provide user-facing outputs through a front-end access layer 340 configured to deliver system functionalities to authorized users via browser-based platforms and mobile applications. The front-end access layer 340 may expose personalized interfaces for family members, caregivers, or clinical care providers, with access rights and available features governed by role-based authentication and permission management protocols.

Through the front-end access layer 340, users may access a variety of system-generated content and interactive tools, including but not limited to:
To-do lists derived from personalized care plans, which may present task assignments, scheduled care activities, and adherence tracking metrics.
ADL engagement tracking modules, which may display real-time or historical records of completed, assisted, or missed activities of daily living (ADLs).
ADL training modules, which may provide caregivers with task-specific instructional content, procedural guidelines, or competency validation tools related to ADL support
Electronic Visit Verification (EVV) workflows, enabling caregivers to log visit start and end times, confirm service delivery, and comply with regulatory reporting requirements.
Micro-bonusing modules, which may offer real-time incentives, acknowledgments, or performance-based rewards to caregivers based on detected care activity, engagement scores, or resident flourishing outcomes.

In some embodiments, the system may further include a caregiver support platform configured to facilitate coordination, resource sharing, and supplemental service provision among multiple caregivers. The platform may include mobile application interfaces allowing caregivers to schedule coverage exchanges, manage time-off periods, and coordinate support during vacations, illness, or shift transitions. In addition to task scheduling and activity prompts, the system may provide access to curated caregiver resources, such as recommended tools, equipment, procedural guides, or best-practice solutions designed to support caregiver well-being and optimize care delivery. Caregiver mobile applications may present structured tools, educational content, product recommendations, or peer-exchange opportunities, enabling caregivers to enhance their service quality, resilience, and professional development.

In some embodiments, authenticated ADL event data may be formatted and transmitted in compliance with applicable regulatory frameworks, including but not limited to Medicaid Electronic Visit Verification (EVV) requirements, eldercare tax credit substantiation protocols, or long-term care accreditation reporting standards. Compliance modules may adapt system outputs to the format, frequency, and certification needs of external regulatory bodies, facilitating auditable, policy-conforming documentation of care activities.

In some embodiments, the front-end access layer 340 may deliver dynamic updates, push notifications, or actionable prompts derived from back-end analytics performed within the system 300, including outputs generated by the care evaluation and planning engine 260 or external analytics services accessible through the external systems and services interfaces 330.

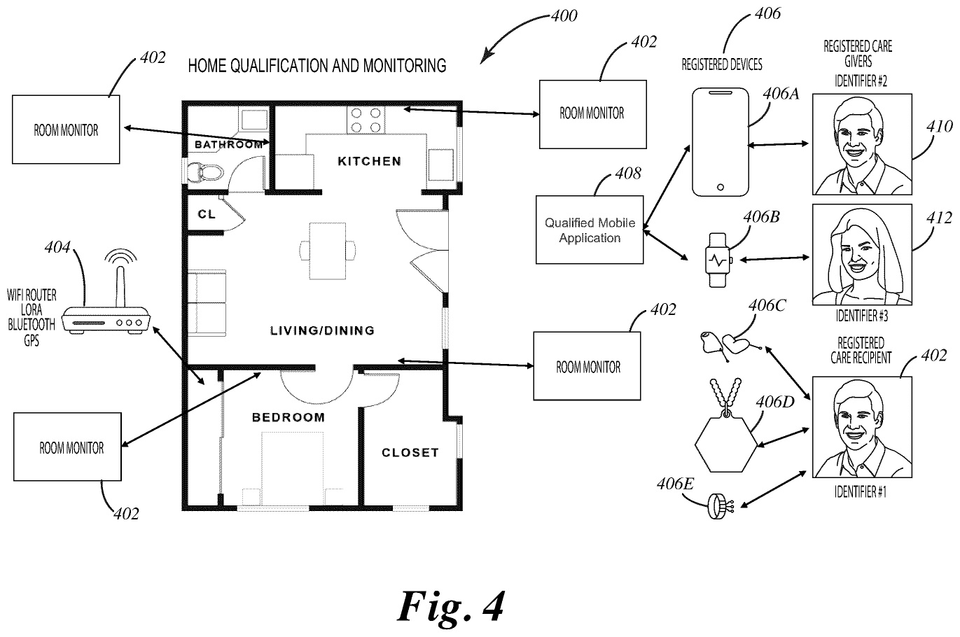

Referring now to FIG. 4, an exemplary residential care monitoring environment 400 is illustrated. The residential care monitoring environment 400 may correspond to a private residence, assisted living unit, or similar care setting configured to support activity detection, environment qualification, and identity-based monitoring of care-related interactions. As shown, the residential layout includes distinct zones such as a bedroom, bathroom, kitchen, living/dining area, and closet, with monitoring devices and communication infrastructure deployed to facilitate comprehensive, multi-zone observation.

In some embodiments, the residential care monitoring environment 400 may form part of a qualified residential monitoring environment 102 as previously described in connection with FIG. 1. The illustrated configuration in FIG. 4 demonstrates one example of how environmental sensors, communication nodes, and registered user devices may be spatially distributed to capture relevant care activities and support authenticated monitoring workflows.

As further shown in FIG. 4, a plurality of room monitors 402 may be deployed within the residential care monitoring environment 400 to detect environmental conditions, user activities, and contextual information within defined zones of the environment. Each room monitor 402 may include one or more sensing or communication modalities, including but not limited to:
low-power wide-area networking (LPWAN) communication modules, such as modules compatible with LoRa protocols;
short-range wireless networking modules, such as WiFi-compatible radios;
proximity-based wireless communication modules, such as Bluetooth Low Energy (BLE) beacons;
radio signal strength monitoring modules, configured to capture received signal strength indicator (RSSI) values;
acoustic event detection modules, configured to capture sound pressure or acoustic waveform patterns;
optical sensing modules, including imaging sensors or video capture devices; and
environmental gas concentration sensors, such as carbon dioxide (CO₂) detectors.

In some embodiments, the room monitors 402 or associated processing modules may be further configured to analyze voice patterns associated with care recipients or caregivers, such as speech-related engagement characteristics or emotional tone indicators. Voice pattern analysis may be used to supplement acoustic event detection, provide additional context for ADL event authentication, or inform personalized care planning and guidance outputs. Privacy-preserving techniques, such as local feature extraction without transmission of raw audio, may be optionally employed to protect user confidentiality.

In some embodiments, room monitors 402 may be statically positioned within specific rooms, such as a bedroom, bathroom, kitchen, or living/dining area, to provide localized sensing coverage aligned with common activities of daily living (ADLs). The specific combination of sensing modalities deployed within a given environment may vary based on monitoring requirements, deployment constraints, or user configuration preferences.

Table 1 illustrates an exemplary deployment of various sensing modalities across typical residential care environment zones. The table outlines how different sensor types including Bluetooth signal strength indicators (RSSI), photodiodes for light detection, passive infrared (PIR) motion sensors, microphones for sound detection, environmental gas sensors such as CO₂ monitors, accelerometers, and air quality sensors may be strategically distributed among residential rooms such as bathrooms, kitchens, bedrooms, and living areas. For each sensor type, the table indicates associated activity or environmental monitoring functions performed within each room. For example, motion sensors may support proximity and activity detection across all monitored zones, while humidity sensors in bathrooms may facilitate detection of showering and bathing activities. This multi-sensor deployment framework enables comprehensive coverage of relevant Activities of Daily Living (ADLs) and environmental conditions within the residential care environment, supporting robust event detection, activity authentication, and health-related monitoring workflows.

TABLE 1
Home Healthcare Hub Locations and Monitoring

| Sensor | Type | Bathroom | Kitchen | Bedroom | Living Room |
|---|---|---|---|---|---|
| RSSI | Bluetooth | Location and | Location and | Location and | Location and |
| | | proximity | proximity | proximity | proximity |
| Light/sunlight | Photodiode | Activity | Activity | Activity | Activity |
| Motion | PIR | Proximity and | Proximity and | Proximity and | Proximity and |
| | | activity | activity | activity | activity |
| Sound | Microphone | Proximity, | Proximity, | Proximity and | Proximity, |
| | | activity, toilet | activity, dish | activity | activity, TV and |
| | | flushing | activity | | radio |
| Humidity | RH | Shower, bathing | Sink activity | Humidity | Humidity |
| Temperature | | Shower, bathing | Temperature | Temperature | Comfort |
| | | | | | selection |
| CO2 | Sensirion | Occupancy and | Occupancy and | Occupancy and | Occupancy and |
| | SCD30 | breathing rate | breathing rate | breathing rate | breathing rate |
| Accelerometer | | Fall event | Fall event | Fall event | Fall event |
| Air quality | Bosch BM E688, | Event tracking | Cooking | Air quality | Air quality |
| | VOC, IAQ, | | | | |
| | CO2 | | | | |
| Door open/close | Magnet/Zigbee | Entry exit, log, | Entry exit, log, | Entry exit, log, | Entry exit, log, |
| | | medication | medication | medication | medication |
| | | access | access | access | access |

Room monitors 402 may operate independently or cooperatively, and may communicate with other system components using one or more wireless communication protocols. Data captured by room monitors 402 may be transmitted to backend processing modules for event authentication, environment qualification, or care interaction tracking, as described in greater detail with respect to FIGS. 1 and 2.

A wireless communication router 404 may be deployed within the environment 400 to provide local networking infrastructure and device connectivity support. In some embodiments, the wireless communication router 404 may be configured to support multiple wireless communication protocols, including but not limited to WiFi, Bluetooth Low Energy (BLE), low-power wide-area networking (LPWAN) technologies such as LoRa, and GPS positioning functionality. The wireless communication router 404 may serve as a network access point for local device communication, a data aggregation hub for transmitting monitoring data to backend processing systems, or a gateway node supporting secure communication with external networks. Placement of the wireless communication router 404 within the environment 400 may be determined based on coverage requirements, physical obstructions, device proximity optimization, or network topology considerations.

The residential care monitoring environment 400 may further include a plurality of registered devices 406 associated with individual caregivers or care recipients. These devices may be configured to emit identification data, collect activity-related information, or facilitate authenticated interactions within the environment 400. In some embodiments, registered devices may include a smartphone 406A, a smartwatch 406B, a hearing aid device 406C, a personal emergency response device 406D, and a ring-based wearable device 406E. Each registered device may be assigned to a specific user identity and configured to communicate with room monitors 402, the wireless communication router 404, or backend processing systems via one or more wireless protocols, including WiFi, Bluetooth Low Energy (BLE), LPWAN technologies such as LoRa, or other communication technologies.

The smartphone 406A and smartwatch 406B, or any other registered devices, may additionally be configured to execute a qualified mobile application 408, which may provide a trusted software framework for emitting identity signals, receiving care-related prompts, logging task completion, or participating in authentication workflows. The qualified mobile application 408 may support background transmission of persistent or dynamic identifiers, engagement with care plan to-do lists, and real-time location or proximity tracking in connection with environmental monitoring data.

In some embodiments, registered devices 406A-406E may perform overlapping functions, such as emitting short-range beacons for location estimation, capturing biometric data, providing emergency signaling capabilities, or affirming presence within specific residential zones. Device registration workflows may bind each device to a registered user identity to enable multi-source authentication, event correlation, and care interaction tracking across the residential monitoring environment.

As further shown in FIG. 4, the residential care monitoring environment 400 may include a plurality of registered user identities corresponding to individuals interacting within the monitored environment. These identities may include one or more registered caregivers 410, 412 and a registered care recipient 414. Each registered user may be associated with identifiers, such as device registrations (e.g., smartphones, wearables, emergency pendants), facial recognition profiles, biometric templates, cryptographic tokens, or other forms of identification.

The registered caregivers 410 and 412 may be professional caregivers, family members, or other authorized personnel responsible for delivering care-related assistance, supervising activities of daily living (ADLs), or responding to alerts within the residential environment. Caregiver identifiers may be dynamically associated with physical presence detection, ADL engagement events, or task completion records as captured by the room monitors 402 and registered devices 406A-406E.

The registered care recipient 414 may correspond to the individual receiving monitored care services within the environment 400. In some embodiments, the care recipient's identifiers may include persistent device associations, facial recognition mappings, biometric monitoring streams, or engagement profiles maintained within backend authentication systems. Registration of the care recipient enables personalized activity tracking, care plan adherence analysis, and context-specific prompting based on real-time environmental observations.

In various embodiments, user registration may occur during system initialization, enrollment workflows, or dynamic updates to accommodate caregiver turnover or changes in care recipient status. The registration framework supports multi-user authentication, privacy-preserving monitoring, and flexible adaptation to evolving care needs within a monitored residential environment.

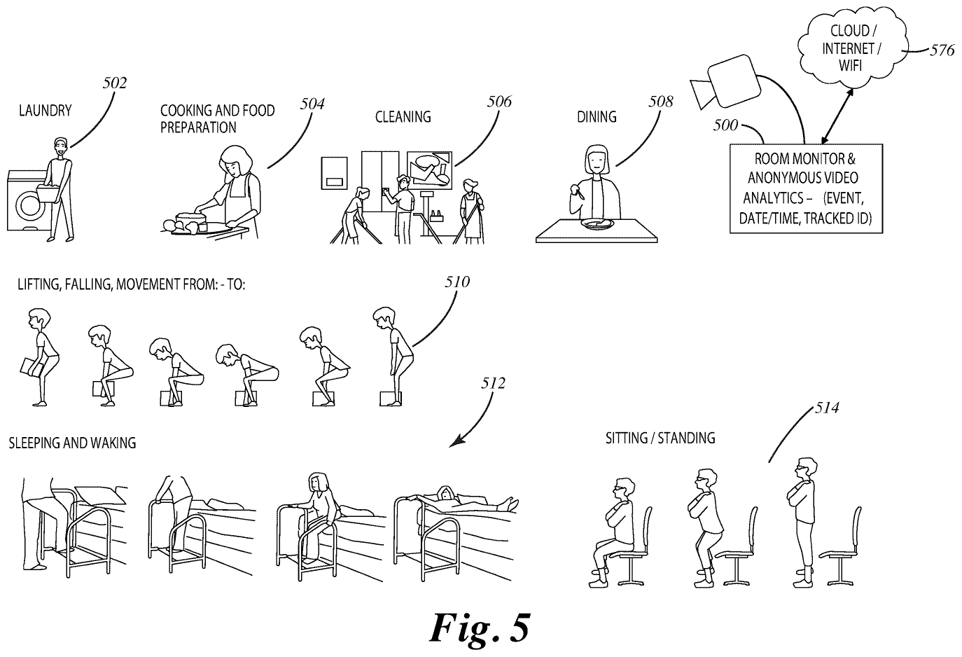

Referring now to FIG. 5, an exemplary embodiment of a room monitoring and anonymous video analytics system 500 is illustrated. The system 500 may include one or more imaging devices, such as cameras or optical sensors, configured to observe physical activities occurring within a residential care environment. In some embodiments, the room monitoring system 500 may be integrated with or communicatively coupled to local processing modules configured to perform real-time activity recognition, posture tracking, movement analysis, or object interaction detection.

The room monitoring and analytics system 500 is configured to detect and classify activities of daily living (ADLs) based on observed motion patterns, positional changes, environmental context, and other non-biometric activity features. Rather than transmitting or storing raw video or identifiable imagery, the system 500 may extract structured event data, including activity type, event timestamp, and a non-biometric tracked identifier correlated to a registered care recipient or caregiver device. This event data may then be transmitted via a communication network 516, such as a local WiFi network or the Internet, to a backend system for authenticated ADL recording, care plan verification, or reporting purposes.

In various embodiments, the room monitoring system 500 may be configured to operate continuously or event-driven, may employ localized edge-processing techniques to enhance privacy, and may flexibly adapt its detection thresholds based on environmental conditions, user mobility patterns, or system configuration preferences.

The room monitoring and anonymous video analytics system 500 may be configured to detect a plurality of activities of daily living (ADLs) based on structured observation of user motion patterns, environmental context, and interaction cues within the residential care environment. Detected activities may include, without limitation, laundry-related activities, cooking and food preparation tasks, cleaning actions, dining behaviors, lifting or falling events, sleeping or waking transitions, and sitting or standing postural changes. Each of these activities may be identified using structured analytics applied to imaging data, movement trajectories, environmental mapping, and corroborating device telemetry.

Laundry activity detection 502 may involve recognizing repetitive motions associated with carrying laundry baskets, operating washing machines or dryers, or folding clothing. Detection algorithms may identify load-carrying postures, appliance interaction gestures, or location transitions associated with laundry zones within the residence. In some embodiments, laundry activity detection 502 may be implemented by analyzing object-carrying sequences inferred from posture shifts and arm movement trajectories captured by optical sensing modules. Repetitive bending, lifting, and load-bearing movement patterns localized within laundry-designated areas may be classified as laundry activities. RSSI and BLE proximity data from registered mobile devices 406A-406E may optionally corroborate user presence during detected motion sequences.

Cooking and food preparation detection 504 may involve identifying activity patterns consistent with food preparation, such as chopping motions, stove interaction, countertop workspace usage, or repetitive hand movements near kitchen appliances. Contextual clues such as location proximity (e.g., presence in kitchen zones) and temporal usage patterns may refine event classification. In some embodiments, optical flow analysis, gesture recognition, or object contact patterns may be leveraged to distinguish cooking activities from non-cooking movement, optionally supplemented by environmental sensor data such as stove usage detectors or thermal imaging cues.

Cleaning activity detection 506 may involve detecting sweeping, mopping, wiping, or dusting activities based on recognized repetitive motions, lower-extremity shifts, and object manipulation patterns indicative of cleaning behaviors. In some embodiments, cleaning detection 506 may be supported by recognizing repeated lateral hand motions near floor or surface planes, combined with low-body postural shifts indicative of cleaning stance. Device proximity, sound patterns (e.g., broom or vacuum acoustics), or location-based activity modeling may further enhance classification fidelity.

Dining activity detection 508 may involve recognizing seated eating behaviors, including table proximity, hand-to-mouth gestures, or utensil manipulation. Dining events may be differentiated from other seated activities through posture analysis, table interaction tracking, or time-of-day contextual alignment. In some embodiments, pose estimation frameworks may identify characteristic forward-leaning motion, repeated hand elevation events toward the mouth region, and lateral hand movements associated with utensil usage, optionally correlated with presence in a known dining area.

Lifting, falling, and movement transition detection 510 may involve monitoring biomechanical sequences associated with lifting objects, bending, falling incidents, or transitions from one body position to another. The system 500 may detect rapid acceleration vectors, posture instability indicators, or failure-to-recover patterns indicative of a fall or adverse event. In some embodiments, lifting and fall detection 510 may utilize time-series movement trajectory modeling, posture deviation thresholds, and recovery motion analysis to distinguish between normal activity transitions and adverse fall scenarios.

Sleeping and waking detection 512 may involve observing movement patterns associated with bed entry, sleep postures, waking transitions, or prolonged immobility within bed zones. Sleep and waking events may be inferred based on horizontal posture recognition, time duration analysis, and coordinated low-movement signatures over extended time windows. In some embodiments, sleeping detection 512 may further incorporate environmental cues such as lighting changes, CO₂ level stabilization, or scheduled sleep-time metadata to enhance event classification accuracy.

Sitting and standing detection 514 may involve detecting transitions between seated and standing postures, including chair usage, balance shifts, and upright locomotion initiation. In some embodiments, sit-to-stand transition detection 514 may be based on posture elevation tracking, vertical displacement rate thresholds, or balance stabilization pattern recognition. Frequency, duration, and smoothness of sit-to-stand transitions may be quantified over time to assess mobility status, physical independence, or early signs of decline.

In certain implementations, the system 500 may apply rule-based heuristics, machine learning classification models, or configurable detection thresholds to identify activity events. Detected activities may be timestamped, assigned to tracked non-biometric identifiers, and transmitted as structured metadata to backend systems 516 for authenticated ADL recording, care plan evaluation, or reporting purposes.

To support privacy preservation while enabling actionable care monitoring, the room monitoring and anonymous video analytics system 500 may be configured to process image or video data locally or within a controlled edge-processing environment. Structured event metadata may be extracted from sensor inputs without retaining or transmitting identifiable visual content. For each detected activity event—such as laundry 502, cooking 504, cleaning 506, dining 508, lifting or falling 510, sleeping or waking 512, or sitting or standing 514—the system 500 may generate a structured record comprising fields such as the detected activity type, event timestamp, a non-biometric tracked identifier, and optionally a confidence score or environmental context marker.

A nonymized identifiers may correspond to non-biometric device IDs, session-based tracking tokens, or pseudonymous user assignments, enabling event correlation and longitudinal analysis without exposing personal identifying information (PII) or biometric features. Raw imagery or sensitive sensor payloads may be discarded, obfuscated, or processed in transient memory structures, thereby preventing persistent storage or external transmission of identifiable media.

Structured event records may be securely transmitted via a communication pathway 516, such as an encrypted WiFi, BLE, or LPWAN connection, to backend processing systems for authenticated ADL recording, care quality evaluation, incident flagging, or care plan compliance monitoring. In various embodiments, event aggregation may occur in real-time, periodically, or adaptively based on system configuration, network conditions, or privacy optimization settings. This architecture allows the system 500 to balance fidelity of care monitoring with enhanced data minimization principles, supporting compliance with privacy regulations and related frameworks governing residential activity monitoring.

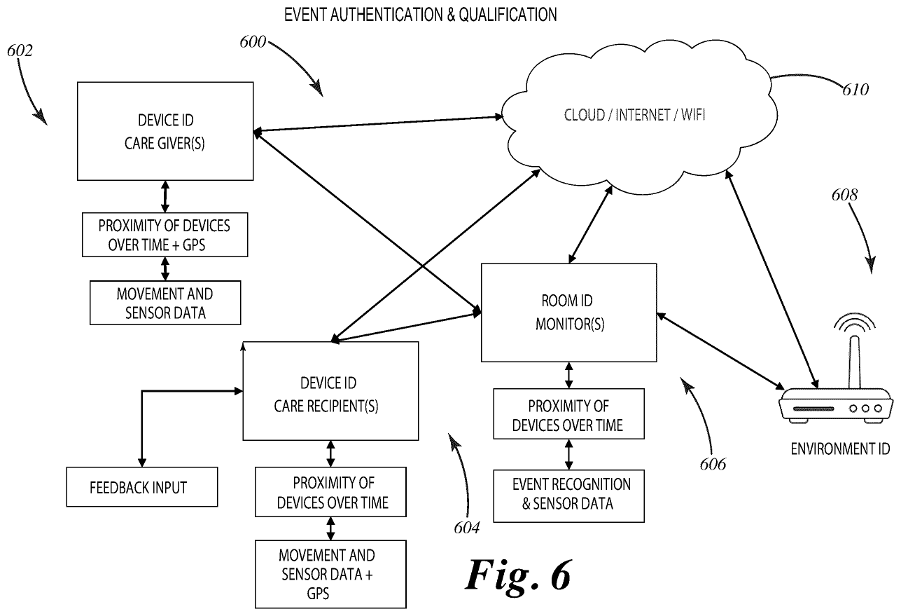

Referring now to FIG. 6, an exemplary embodiment of an event authentication and qualification framework 600 is illustrated. The framework 600 is configured to correlate data streams originating from multiple independent sources within a monitored residential care environment to authenticate detected activities of daily living (ADLs) and qualify care-related events. In the illustrated embodiment, the event authentication system leverages inputs from caregiver devices 602, care recipient devices 604, room monitors 606, and environment devices 608. These inputs may be processed cooperatively or asynchronously to validate that observed activities satisfy predefined authentication criteria, thereby generating structured event records for downstream care plan evaluation, compliance tracking, or care quality monitoring.

The event authentication and qualification framework 600 may be integrated with a backend processing system 610, such as a cloud-based or distributed network infrastructure, to perform higher-level correlation, scoring, and recording of qualified events. In some embodiments, the framework 600 may operate continuously, periodically, or event-driven, depending on system configuration, network availability, and operational policies established for the residential monitoring deployment.

By synthesizing multiple independent data sources, the system 600 can enhance reliability, reduce false positives, and enable privacy-preserving care authentication without reliance on a single sensing modality. Events may be authenticated based on factors such as physical activity detection, device proximity trends, zone occupancy analysis, and environmental context markers, thereby supporting nuanced care verification workflows aligned with individualized care plans.

Table 2 illustrates exemplary reconstruction and tracking strategies for assisted activities within the residential care environment. The table summarizes how multiple sensing modalities including proximity detection, activity monitoring, imaging analytics, environmental sensor measurements, and time log records may be selectively leveraged across different system implementations to reconstruct and authenticate a variety of Activities of Daily Living (ADLs). Assisted events such as food preparation, eating, cleaning, hygiene routines, medication intake, and mobility transitions are mapped against sensing input types that contribute to reliable event recognition under varying implementation configurations. In some embodiments, different system implementations may incorporate different sets of sensing modalities. For example, Implementation 1 may utilize a basic set of modalities (“X”), while Implementation 2 and Implementation 3 incorporate expanded sensing combinations (“XX” and “XXX”, respectively) to achieve enhanced authentication fidelity. This implementation-based reconstruction framework supports deployment flexibility, allows customization based on care recipient needs or monitoring environment characteristics, and enhances robustness against variable sensing conditions. In some embodiments, additional system implementations utilizing alternative combinations of sensing modalities may be supported beyond those explicitly illustrated in Table 1, and the examples provided herein are not intended to be limiting.

TABLE 2
Reconstruction and Tracking of Events

| | | | | | Time |
|---|---|---|---|---|---|
| Assisted Events | Proximity | Activity | Imaging | Sensors | Log |
| Food preparation | X | X | XXX | XX | X |
| Eating | X | X | XXX | XX | X |
| Cleaning | X | X | XXX | XX | X |
| Laundry | X | X | XXX | XX | X |
| Brushing/getting ready | X | X | XXX | XX | X |
| Changing bedding | X | X | XXX | XX | X |
| Waking | X | X | XXX | XX | X |
| Sleeping | X | X | XXX | XX | X |
| Taking medication | X | X | XXX | XX | X |
| Using toilet | X | X | XXX | XX | X |
| Bathing | X | X | XXX | XX | X |
| Sitting/getting up | X | X | XXX | XX | X |

Implementation 1 X
Implementation 2 XX
Implementation 3 XXX

The event authentication and qualification framework 600 may incorporate device-based data streams from both caregiver devices 602 and care recipient devices 604. Each device may include a plurality of sensing capabilities and may emit structured metadata to support activity verification.

The caregiver device 602 may include features such as:
A device identifier, such as a MAC address, cryptographic token, or assigned system registration number, enabling persistent or session-based association of sensed data with a particular caregiver.
Proximity tracking data, based on Bluetooth Low Energy (BLE) beacons, WiFi signal strength mapping, or similar short-range wireless positioning techniques, to monitor caregiver presence within specific zones of the residential environment over time.
Global navigation satellite system (GNSS) data, such as GPS coordinates, providing broader location awareness to supplement proximity determinations, particularly in larger or multi-zone deployments.
Movement and sensor telemetry, such as accelerometer readings, gyroscopic motion data, posture detection signals, or derived motion trajectories, capturing physical activity patterns and interaction signatures associated with care delivery.

Similarly, the care recipient device 604 may include corresponding sensing capabilities and may generate structured outputs including proximity, positional, and movement data streams. These data may enable differentiation between caregiver-driven activities and care recipient-initiated behaviors. In some embodiments, relative proximity measurements, co-location event patterns, or coordinated motion profiles may be analyzed to validate care interactions, detect independent versus assisted ADL completions, or identify deviations from expected routines.

Data emitted from caregiver devices 602 and care recipient devices 604, including device-generated identifiers and sensed telemetry, may be transmitted directly to backend processing infrastructure 610 or optionally pre-processed locally by room monitors 606 or residential network gateways associated with environment devices 608, depending on deployment topology and privacy management policies.

The framework 600 may further incorporate environmental sensing inputs from room monitors 606 and infrastructure nodes such as environment devices 608. These additional sources provide spatial, contextual, and environmental evidence to support multi-modal event verification.

Room monitor 606 inputs may include:
Event recognition data, such as detected activities based on optical sensing (e.g., movement trajectories, posture classification, object interaction analysis), acoustic sensing (e.g., sound pattern matching, voice activity detection), or environmental monitoring (e.g., CO₂ concentration, temperature changes, or appliance usage detection).
Proximity tracking information, derived from passive or active sensing modalities, such as Bluetooth Low Energy (BLE) beacons, WiFi triangulation, or short-range radio signal mapping, enabling zone-based presence detection for registered devices.
Timestamped zone occupancy records, reflecting the presence or absence of care recipients or caregivers within specific monitored zones (e.g., bedroom, kitchen, living/dining area) over defined time intervals.
Anonymized activity metadata, including event labels, confidence scores, and pseudonymous tracked identifiers extracted without persistent retention of sensitive raw imagery.

The environment devices 608 may correspond to wireless communication routers, mesh network nodes, or other infrastructural elements facilitating residential connectivity and location anchoring. These devices may be used to associate sensed activity streams with specific environments, optimize network-based localization, and support environmental context validation during event authentication processes.

Inputs from room monitors 606 and environment devices 608 may be processed locally or transmitted to backend systems 610 in real-time, periodic, or adaptive aggregation workflows. These environmental data sources, in conjunction with caregiver and care recipient device data, enable the system 600 to perform multi-source event authentication with increased reliability and environmental awareness.

In some embodiments, the system may further support secondary caregiver tracking, enabling monitoring of care interactions performed by both primary and secondary care providers within the residential environment. Caregiver roles, activities, eligibility levels, and associated qualification parameters may be dynamically correlated to registered caregiver identifiers, facilitating compliance tracking and care validation across diverse caregiving personnel. The system may additionally authenticate medication-related Activities of Daily Living (ADLs), including tracking of medication access events, caregiver-assisted medication delivery, and care recipient acknowledgment of medication consumption to provide proof of delivery. Environmental monitoring features may include analysis of room-specific CO₂ levels and ventilation patterns in combination with detected dining activities to infer metabolic activity levels, as well as humidity and water flow sensing associated with hygiene-related activities such as bathing and showering. The system may further track non-clinical care activities, including cooking, feeding, laundry management, bedding changes, and cleaning activities, using proximity sensing, activity recognition, and environmental signal analysis. Integration with third-party wearable devices or mobile APIs, such as Oura rings or smartwatches, may support enhanced monitoring of physical activity, exercise routines, and sleep transitions. Sleep-related ADLs may also incorporate environmental data from CO₂ sensors, bed occupancy sensors, or CPAP device integrations to track sleep quality and nighttime caregiving exchanges. Anonymous image processing techniques may be applied to support non-intrusive detection of care recipient activity and caregiver participation, enabling AI-based activity recognition and caregiver presence validation without the storage or transmission of identifiable image data.

In some embodiments, the system may be configured to monitor, authenticate, and evaluate both primary and secondary Activities of Daily Living (ADLs) within the residential care environment. Primary ADLs may include hygiene, feeding, dressing, mobility, and medical compliance activities, while secondary ADLs may include environmental maintenance, food preparation, laundry handling, exercise facilitation, and social engagement support. The system may integrate data from a wide range of devices, including but not limited to smartwatches (e.g., Apple Watch), biometric monitoring rings (e.g., Oura Ring), emergency alert devices, environmental sensors (e.g., CO₂ monitors), smart beds, CPAP machines, assistive scooters, walkers, lift chairs, shower chairs, and exercise equipment. Data from such devices may be collected via APIs, Bluetooth connections, or IoT-enabled networks. In some embodiments, blockchain or distributed ledger architectures may be utilized to secure and verify authentication records for care events, enabling tamper-resistant audit trails and care compliance tracking. The system may further include mechanisms for ranking or evaluating secondary care provider performance based on authenticated engagement in support activities, environmental maintenance tasks, and care recipient interaction metrics. This expanded ecosystem enables comprehensive validation of holistic care delivery and supports incentive, feedback, or accountability frameworks for professional and family caregivers.

Inputs received from caregiver devices 602, care recipient devices 604, room monitors 606, and environment devices 608 may be correlated and authenticated within backend processing system 610. In some embodiments, backend system 610 may form part of the ADL authentication system 120, the care evaluation and planning engine 260, or the broader care business operating system 300 described previously in connection with FIGS. 1 through 3.

The backend event authentication framework may apply a multi-source event verification model in which detected activities are authenticated based on three primary components:
Event Recognition: Identification of a candidate activity, such as laundry folding, food preparation, or surface cleaning, based on device data and environmental observations.
Time Correlation: Assignment of a specific timestamp or bounded time interval during which the candidate activity occurred, ensuring temporal precision for event recording
Independent Verification Points: Validation of the candidate activity using at least two independent streams of corroborating evidence (e.g., caregiver movement telemetry 602 and room monitor event recognition 606).

An activity may be classified as a qualified event when it is supported by:
A recognized event type,
A timestamped occurrence window, and
Two or more independent streams of corroborating evidence.

This structured verification approach enhances the robustness of care authentication, reduces false-positive classifications, and provides strong evidentiary support for care plan compliance tracking, ADL engagement scoring, and personalized care plan adjustment workflows.

Qualified event records may include structured fields such as event type, timestamp, associated tracked identifiers, confidence scores, and environmental context metadata. Such records may be encrypted and stored in secure backend repositories, available for downstream processing or care optimization.

Byway of example, FIG. 6 illustrates how a typical qualified care event, such as a laundry assistance session, may be authenticated. In this scenario, a caregiver equipped with a registered caregiver device 602 performs laundry-related tasks within a monitored residential environment.

During the care session, the caregiver device 602 may emit proximity signals and motion telemetry indicating presence and activity within the laundry zone, while room monitor 606 may detect corresponding event patterns, such as repetitive bending, appliance interaction, and folding gestures.

The backend system 610 may correlate the data streams, confirming:
Event Recognition (detected laundry activities),
Time correlation (sustained engagement over a specified window),
Independent verification (caregiver device movement plus room monitor event recognition).

Upon satisfying these authentication criteria, the observed activities may be classified as a qualified laundry care event. Event metadata, including the event type (laundry assistance), timestamp, caregiver association, duration, and supporting confidence measures, may be recorded in backend systems.

Qualified event records may be used for multiple purposes, including validating completion of care plan tasks, updating ADL engagement histories, generating caregiver coaching feedback, and facilitating care plan optimization.

Environmental context data from environment devices 608, such as proximity stability, gateway node continuity, or room monitor coverage adequacy, may be used to enhance event confidence scoring and to detect potential infrastructure degradation over time.

This event authentication and qualification framework 600 illustrates how multi-source sensing, distributed verification, and structured event recording enable robust, privacy-preserving care monitoring within residential care settings.

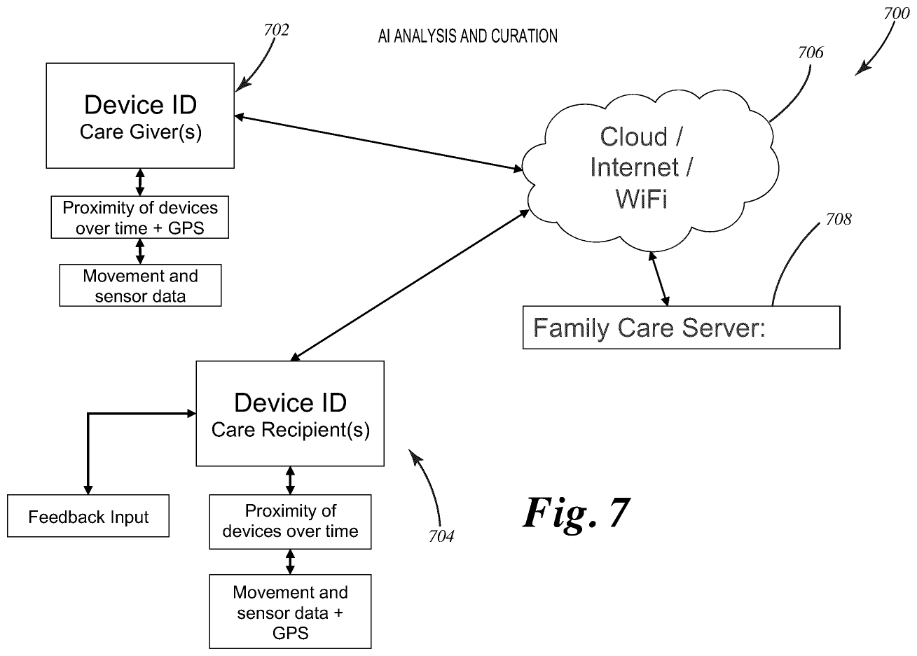

Referring now to FIG. 7, an exemplary embodiment of an AI analysis and curation framework 700 is illustrated. The framework 700 is configured to receive structured event metadata, proximity data, and sensor data streams originating from caregiver devices 702 and care recipient devices 704, as previously described with respect to FIGS. 5 and 6. These device-originated data streams may be transmitted across a cloud-based or distributed communication infrastructure 706 to a backend processing platform referred to herein as a family care server 708.

The family care server 708 is configured to perform higher-level analytic functions associated with care plan curation, coaching generation, compliance tracking, and residential environment qualification. In some embodiments, the family care server 708 may form part of the broader ADL authentication system 120, the care evaluation and planning engine 260, or external family care coordination servers 160F as introduced in FIGS. 1 through 3. Processing operations within the server 708 may be performed continuously, periodically, or adaptively based on event detection thresholds, caregiver intervention cycles, or predefined care plan review schedules.

Through the integration of device data, event histories, and environmental assessments, the AI analysis and curation framework 700 enables dynamic, personalized refinement of care workflows, caregiver feedback mechanisms, and care environment assessments, thereby supporting ongoing optimization of residential elder care engagements.

The AI analysis and curation framework 700 may receive structured input data streams from both caregiver devices 702 and care recipient devices 704. These devices, which may include smartphones, wearable sensors, proximity tags, or specialized care monitoring hardware, are registered to respective users within the residential care environment and operate in conjunction with previously described monitoring systems.

Each device stream 702, 704 may include a variety of structured data elements, including but not limited to:
Movement and sensor telemetry, such as accelerometer readings, gyroscope data, posture classification outputs, or derived motion trajectories, which can be used to infer user activities, mobility patterns, and interaction sequences.
Proximity tracking information, collected through mechanisms such as Bluetooth Low Energy (BLE) beacon ranging, WiFi triangulation, or short-range radio positioning, which can establish spatial relationships between users and environmental zones over time.
Session-based or persistent device identifiers, used to associate sensed activity data with specific registered users while preserving anonymity where appropriate through pseudonymous or cryptographically secure tracking tokens.
Feedback input signals, in the case of care recipient devices 704, which may encompass active responses to system prompts, passive behavioral cues inferred from device usage patterns, or explicit self-reported activity confirmations.

In various embodiments, the data streams from caregiver devices 702 and care recipient devices 704 may be transmitted to backend processing systems either in real time, near-real-time bursts, or scheduled batch uploads, depending on system configuration, network availability, and privacy management policies.

The integration of these multi-modal, timestamped, and spatially contextualized data streams enables the family care server 708 to perform higher-order analytics, including care plan validation, care interaction assessment, and residential qualification evaluation, as described in further detail below.

The family care server 708 is configured to perform a range of higher-order analytic and curation functions based on the structured input data streams received from caregiver devices 702 and care recipient devices 704. In particular, the server 708 may be configured to apply machine learning models, rule-based decision engines, heuristic scoring algorithms, or a combination thereof to dynamically manage and personalize care plans for the monitored environment.

Key functional capabilities of the family care server 708 may include:
Curation of the Care Plan: The server 708 may analyze authenticated ADL event records, device interaction histories, and observed engagement patterns to generate, update, or refine individualized care plans. Plan curation may involve adjusting task frequency, care support intensity, or goal prioritizations based on longitudinal engagement metrics and event qualification trends.
Generation of Curated Coaching Plans for Caregivers and Family M embers: Based on identified care engagement strengths, gaps, or emerging needs, the server 708 may create coaching plans targeting specific behavioral improvements, skill development, or attention areas. Coaching plans may include recommended ADL support activities, motivational interventions, or micro-learning content designed to enhance care delivery effectiveness.
Validation of Care Plan Adherence and Sufficiency: The server 708 may validate whether the activities logged through caregiver devices 702, care recipient devices 704, and room monitoring infrastructure are consistent with care plan goals and minimum standards. Validation may involve threshold checks (e.g., activity completion rates), sequence analysis (e.g., confirming order of task performance), or proximity confirmation (e.g., verifying caregiver presence during dependent ADLs).
Recognition of Care Plan Adjustability: By applying dynamic weighting algorithms, the family care server 708 may detect when an existing care plan becomes insufficient, overly burdensome, or otherwise misaligned with the observed behavior or condition changes of the care recipient. Detection triggers may include ADL disengagement patterns, environmental constraint changes, or caregiver-reported difficulties.

In various embodiments, curated care plans, coaching modules, and validation results may be rendered through family-facing mobile applications, caregiver portals, or clinician dashboards, thereby enabling informed, data-driven adjustments to residential care engagements. Plan updates may occur continuously, on a scheduled review cycle, or adaptively based on predefined triggering conditions configured within the backend system 708.

In addition to care plan curation and coaching functions, the family care server 708 may be configured to perform assembly of activity sequences and residential environment qualification analysis based on correlated event and sensor data.

The assembly of activity sequences involves analyzing temporally ordered event records, proximity transitions, and motion trajectories to reconstruct care interactions and daily living workflows. For example, the server 708 may sequence detected ADL events—such as standing from bed, walking to the kitchen, preparing food, and dining—to confirm the continuity, timing, and completeness of expected routines. Sequence analysis may leverage time gap thresholds, spatial movement mapping, and device co-location tracking to validate the authenticity and sufficiency of care engagements.

In some embodiments, the server 708 may also infer behavioral health indicators, such as changes in activity cadence, increased rest periods, or shortened mobility routes, based on deviations in historical activity sequences compared to baseline models. Such insights may trigger notifications to caregivers, family members, or clinicians for further review or intervention.

The home qualification analysis function assesses whether the monitored residential environment provides sufficient coverage, connectivity, and monitoring fidelity to reliably support authenticated care tracking. The family care server 708 may evaluate:
Sensor and monitor coverage: Confirming that all critical zones (e.g., bedroom, bathroom, kitchen) are equipped with functional monitoring devices capable of detecting target ADL activities.
Device communication integrity: Verifying that registered caregiver and care recipient devices maintain consistent proximity tracking and data transmission capability across the residential environment.
Environmental dynamics: Assessing changes such as new obstructions, remodeling, or network signal degradation that could compromise monitoring accuracy.

If deficiencies are detected, the system may generate a qualification recommendation, which may include suggestions to add monitoring nodes, reconfigure device placements, optimize network topologies, or, in extreme cases, flag the environment as non-qualifiable for certain classes of care monitoring certifications.

By performing continuous or periodic home qualification assessments, the family care server 708 ensures that care authentication reliability remains high and that system performance degradation is identified proactively, thereby supporting sustainable and compliant care monitoring over time.

By way of example, the family care server 708 may apply its activity sequence assembly and home qualification functions in the following representative scenario. During normal operation, a registered care recipient may typically follow a predictable morning routine involving sleeping in the bedroom, rising and standing, walking to the kitchen, and preparing breakfast, as verified by historical sequence patterns constructed from device proximity data 704 and room monitor activity records 606.

Over time, however, the family care server 708 may detect a deviation from the established sequence. Specifically, the care recipient device 704 may exhibit prolonged inactivity in the bedroom, absence of standing or walking transitions, and missing event markers typically associated with kitchen activities. In addition, room monitor data 606 may indicate increased CO₂ levels or lack of detected movement within expected time windows.

In response to detecting such a deviation, the family care server 708 may initiate a sequence alert workflow, flagging the missing events as potential indicators of mobility deterioration, illness, or sensor failure. Concurrently, the server 708 may perform a residential qualification reassessment, evaluating whether network coverage gaps, device battery failures, or environmental layout changes may have contributed to the anomaly.

Based on the integrated assessment, the system may recommend one or more interventions, such as immediate caregiver contact, device repositioning, environmental reconfiguration, or clinician review of the care recipient's status. In some embodiments, adaptive care plan modifications may also be generated automatically, such as increasing monitoring frequency or prompting direct caregiver check-ins during at-risk periods.

This scenario illustrates how the AI analysis and curation framework 700 can leverage real-time event assembly and environment qualification processes to support proactive, personalized care adjustments and to maintain high reliability in residential monitoring deployments.

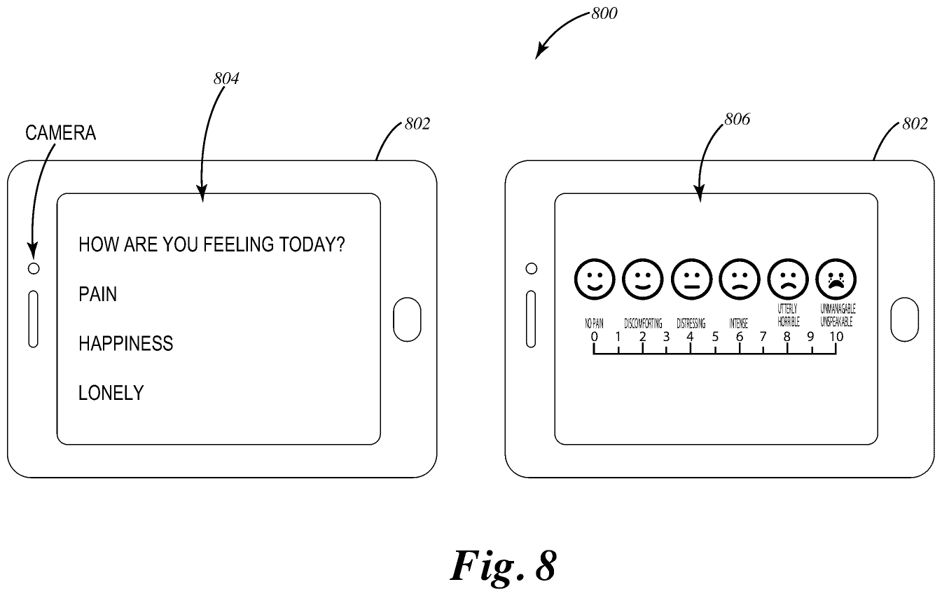

Referring now to FIG. 8, an exemplary embodiment of a feedback collection and analysis framework 800 is illustrated. The framework 800 is configured to capture structured input from care recipients through a feedback collection device 802, as well as to optionally capture passive observational cues through associated sensing components. In the illustrated embodiment, the feedback collection device 802 may include a tablet, mobile computing platform, or dedicated care monitoring terminal equipped with an input interface (e.g., touchscreen or physical button(s)). The device may include a camera, and an audio interface such as a speaker and/or microphone.

FIG. 8 illustrates two representative user interface states rendered by the feedback collection device 802 during an interactive feedback session. In the first state 804, the device 802 presents a category selection interface in which the care recipient may select a feedback category, such as pain, happiness, or loneliness, using the input interface. In the second state 806, following category selection, the device 802 presents a scale or rating input interface corresponding to the selected category. For example, if the user selects “pain,” a visual analog scale ranging from “no pain” to “unimaginable pain” may be displayed, allowing the user to indicate severity via a touch input.

In addition to direct user interaction, the feedback collection device 802 may be configured to capture passive observational data through its integrated camera. Such passive inputs may include facial expression analysis, gesture recognition, or behavioral pattern observation, enabling supplemental inference of emotional state, discomfort levels, or cognitive engagement without requiring explicit user action.

Collected structured feedback, whether direct or passive, may be anonymized, structured into metadata records, and transmitted securely to backend processing systems for integration into care evaluation workflows, coaching plan generation, or real-time intervention escalation procedures, as described in connection with FIGS. 2 and 7.

The feedback collection device 802 may include any suitable computing platform capable of rendering dynamic user interfaces, capturing user input, and acquiring passive observational data. In the illustrated embodiment, the feedback device 802 is depicted as a tablet-style device, but other form factors, such as smartphones, dedicated kiosks, or integrated home automation terminals, may also be employed.

The feedback device 802 may incorporate the following features:
Input Interface: A touchscreen display and/or physical input buttons configured to receive direct responses from the care recipient. Input events may include category selections, scale ratings, freeform text entries, or acknowledgment of system prompts.
Camera Module: An integrated imaging sensor configured to capture visual data streams, such as facial expressions, gaze direction, head movements, or gross motor gestures. In some embodiments, the camera module may be selectively activated based on system prompts, session triggers, or user consent configurations.
Audio Interface: A speaker configured to provide auditory prompts, question readouts, or motivational encouragements to support accessibility and engagement, and optionally a microphone configured to receive verbal responses or passive audio context (e.g., vocal tone analysis).
Communication Module: Wireless networking capabilities, such as WiFi, Bluetooth, or LPWAN, enabling secure transmission of collected structured feedback and metadata to backend processing infrastructure for further evaluation and integration into care workflows.
Privacy and Security Features: In some embodiments, the feedback device 802 may implement on-device encryption, session-based authentication, or data minimization techniques (e.g., local extraction of structured features without retention of raw images or audio recordings) to enhance user privacy and regulatory compliance.

The hardware and sensing capabilities of the feedback device 802 enable flexible, multimodal interaction models, supporting both direct engagement by the care recipient and passive inference of emotional or physical well-being indicators, as further described below.

As shown in FIG. 8, the category selection interface 804 represents a first interaction stage rendered by the feedback collection device 802. In this interface, the care recipient is presented with a selectable list of feedback categories, each corresponding to an aspect of emotional, physical, or psychological well-being.

Example categories may include, without limitation:
Pain: Reflecting current physical discomfort or symptom intensity.
Happiness: Reflecting positive emotional states or general mood.
Loneliness: Reflecting perceived social isolation or desire for interaction.

Additional or alternative categories may also be configured depending on care plan requirements, caregiver input, clinical protocols, or user preferences. For example, categories such as anxiety, fatigue, energy level, or cognitive clarity may also be included in some embodiments.

The care recipient may interact with the category selection interface 804 through touchscreen taps, button presses, voice selection (if equipped with audio recognition capabilities), or assisted input modes configured to accommodate users with dexterity or cognitive impairments.

Upon selection of a category, the feedback collection device 802 may transition to a corresponding rating input interface 806, as described below, enabling the user to provide a finer-grained self-assessment related to the selected category.

In some embodiments, the category selection process may also trigger passive observational routines using the device camera—for example, capturing baseline facial expression snapshots during selection to supplement structured user input with passive emotional state indicators.

Following category selection via the category selection interface 804, the feedback collection device 802 may transition to a scale or rating input interface 806. In this interface, the care recipient is presented with a structured input mechanism for providing a finer-grained self-assessment related to the selected category.

The scale or rating input interface 806 may present visual analog scales, such as a continuum of icons ranging from smiling to frowning faces, numeric ratings (e.g., 0 to 10), or sliding bar interfaces indicating degrees of intensity, satisfaction, or discomfort. In some embodiments, discrete option buttons may be presented, offering labeled selections such as “No Pain,” “Mild Pain,” “Moderate Pain,” “Severe Pain,” and “Unbearable Pain,” each mapped internally to structured numerical values. To support accessibility, the input interface may also incorporate enlarged selection targets, voice feedback prompts, or alternative interaction modes designed to accommodate users with visual, motor, or cognitive impairments.

Upon receiving the user's input selection on the rating interface 806, the feedback collection device 802 may generate a structured feedback record. This record may include the selected category (e.g., Pain), the selected rating (e.g., 7/10), a timestamp of the feedback entry, and optionally passive observational metadata, such as concurrent facial expression confidence scores derived from the device's camera module. The structured record may also include a device identifier, or a pseudonymous session ID associated with the care recipient to facilitate secure association with user profiles without exposing personally identifiable information.

Structured feedback records may be encrypted locally and transmitted securely to backend processing systems for integration into care evaluation workflows. In various embodiments, collected feedback may influence coaching plan adjustments, trigger clinician alerts, update baseline health indicators, or augment care plan personalization efforts, as described previously in connection with FIGS. 2 and 7. Longitudinal monitoring of feedback records may also be performed, enabling detection of trends, anomalies, or escalation patterns that warrant proactive care interventions or system alerts.

In embodiments where the feedback collection device 802 utilizes its integrated camera to capture passive observational data, such as facial expressions, gaze direction, or gross motor gestures, privacy-preserving techniques may be implemented to ensure user confidentiality and compliance with applicable privacy regulations. Raw image or video data captured during feedback sessions may be processed locally on the device 802, with only structured metadata—such as emotional confidence scores, head pose classifications, or gesture detection flags—being retained or transmitted to backend systems.

In some configurations, passive sensing features may be selectively enabled or disabled based on user consent, caregiver configuration, or regulatory mandates applicable to the deployment environment. Users or caregivers may be provided with interface controls to manage camera activation, data retention periods, or anonymization settings.

Data anonymization strategies may further include immediate discarding of raw visual payloads following feature extraction, encryption of structured outputs prior to network transmission, and association of observational metadata with pseudonymous session identifiers rather than directly identifiable user profiles. These privacy enhancement mechanisms enable the feedback collection device 802 to support passive emotional state inference and engagement monitoring while maintaining strict adherence to data protection principles and related frameworks.

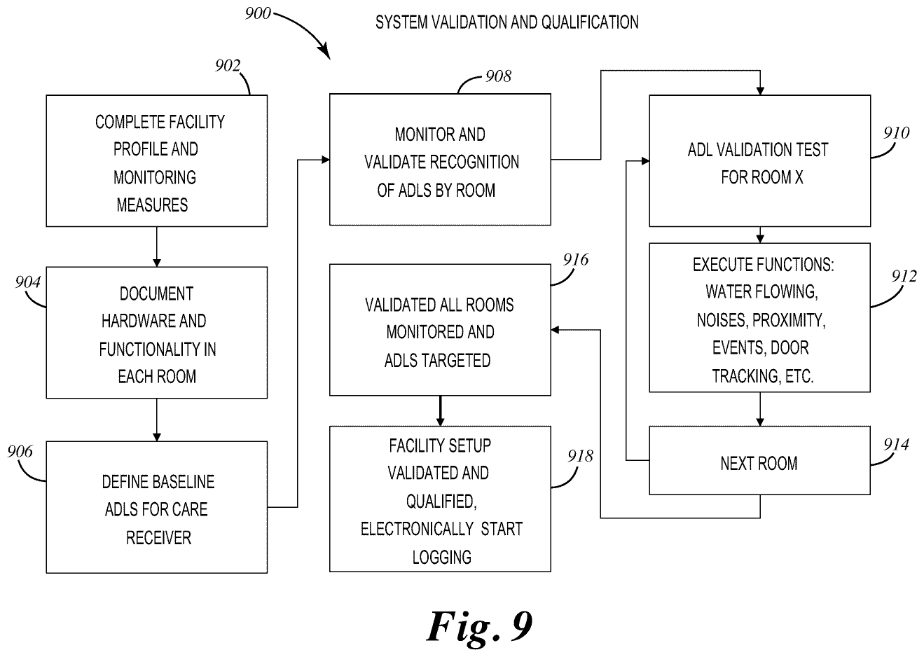

Referring now to FIG. 9, an exemplary embodiment of a system validation and qualification framework 900 is illustrated. The framework 900 is configured to confirm that the residential care environment has been appropriately configured, instrumented, and validated to support authenticated detection of activities of daily living (ADLs) prior to the initiation of formal care monitoring operations.

The validation and qualification process ensures that the residential care monitoring environment meets a defined set of operational readiness criteria prior to initiating formal care logging. Specifically, the process verifies that all intended zones within the residential environment have been equipped with appropriate monitoring hardware positioned to enable effective activity detection; that the installed monitoring systems are capable of reliably detecting the activities of daily living (ADLs) specified within the care recipient's baseline care plan; that the environmental sensing fidelity, communication stability, and data transmission reliability satisfy predetermined operational thresholds; and that the system as a whole is capable of securely initiating authenticated event logging, structured reporting, and care compliance workflows in accordance with system deployment standards and care quality assurance practices.

In the illustrated embodiment, the validation and qualification process 900 proceeds through a series of structured stages, beginning with facility profiling and monitoring documentation, continuing through per-room ADL validation testing, and concluding with facility certification and activation of ongoing logging operations.

Each step in the validation framework 900 is modular and may be adapted or repeated depending on deployment scale, sensor configurations, or care plan complexity. In some embodiments, the validation process may be partially or fully automated, utilizing sensor self-check protocols, synthetic activity generation, or system-prompted test scenarios to minimize manual intervention and accelerate environment readiness certification.

The structured validation approach illustrated in FIG. 9 ensures that care monitoring environments meet minimum quality, coverage, and reliability standards before live care data acquisition begins, thereby enhancing care quality assurance, system integrity, and compliance with care monitoring regulations and standards.

The validation and qualification framework 900 may begin with a facility profiling stage 902, during which a comprehensive profile of the residential care environment is created. This profile may include structural layouts, zone designations, network infrastructure schematics, and identification of care-relevant physical spaces, such as bedrooms, kitchens, bathrooms, and living areas. The profiling process may be based on facility blueprints, manual walkthrough surveys, or automated spatial mapping techniques utilizing environmental sensors or mobile device scans.

Following facility profiling, a documentation stage 904 may be conducted to capture detailed information regarding monitoring hardware deployed within each room or zone. This documentation process may include recording the type, placement, orientation, and functional capabilities of each installed monitoring device, such as room monitors, environmental sensors, proximity beacons, and network access points. Functional attributes documented at this stage may include sensor modalities supported (e.g., optical, acoustic, CO₂ sensing), communication protocols utilized (e.g., WiFi, BLE, LoRa), and any hardware-specific calibration or configuration settings relevant to activity detection.

The facility profile and hardware documentation collectively provide a foundational dataset upon which downstream validation, qualification, and care plan targeting workflows are based. In some embodiments, this initial environmental modeling may also enable automated validation scripts, synthetic test event generation, or predictive analysis of monitoring coverage gaps prior to initiating live care operations.

Upon completion of facility profiling and hardware documentation, the validation and qualification framework 900 may proceed to a room-based monitoring validation stage, beginning with monitoring configuration validation 908. In this stage, each designated room or zone within the residential care environment is individually evaluated to confirm its readiness to support authenticated detection of the activities of daily living (ADLs) targeted in the care recipient's baseline care plan.

For each room, an ADL validation test 910 may be performed. During the validation test, synthetic or actual care-related activities may be executed within the room to assess the operational readiness of the installed monitoring devices. Simulated or prompted activities may include, without limitation, water flow detection from plumbing fixtures, sound pattern recognition associated with movement or appliance usage, proximity tracking of device-bearing individuals, door opening and closing events, and other care-relevant environmental interactions.

As shown in FIG. 9, functions such as water flow monitoring, noise event detection, environmental sensing reactions, and proximity logging may be triggered and evaluated during the ADL validation test 912. These test events verify that the installed monitoring hardware captures, classifies, and transmits event metadata as expected, with sufficient accuracy, reliability, and temporal fidelity to support downstream care verification workflows.

Following successful validation of each room, the validation workflow may proceed to the next room 914, repeating the ADL validation testing cycle until all monitored zones have been assessed. In some embodiments, room validation results may be logged, timestamped, and associated with the facility profile to support historical tracking, audit trail generation, and system performance monitoring over time.

By performing structured, per-room ADL validation testing, the system ensures that environmental monitoring coverage is robust, contextually accurate, and aligned with individualized care plan targets prior to initiation of live care monitoring.

Upon successful completion of room-by-room monitoring validation, the system validation and qualification framework 900 advances to a final certification stage 916. At this stage, the system verifies that all designated rooms have passed ADL validation testing, that no critical monitoring gaps remain, and that environmental sensing coverage and event detection performance meet established quality assurance thresholds.

The final facility validation 916 may include automated cross-checks against the facility profile generated during initial setup, ensuring that all zones marked as critical for care tracking have been fully validated. Summary reports may be generated, documenting the validation status of each room, sensor node performance metrics, and any noted exceptions or configuration advisories.

Once facility validation has been completed and certified, the system may proceed to initiate electronic logging activation 918. Activation of care monitoring involves transitioning the backend systems from test mode to live operational mode, enabling authenticated event recording, care plan compliance tracking, and caregiver engagement monitoring based on real-world data streams.

In some embodiments, activation of electronic logging may be gated by administrative review and signoff, such as by a facility manager, care coordinator, or clinician responsible for oversight. Alternatively, activation may occur automatically upon successful validation of all required monitoring zones, subject to predefined system policy settings.

This structured validation and certification of the care monitoring environment prior to logging activation ensures that residential monitoring systems operate with high reliability, that care tracking workflows are grounded in validated sensing infrastructure, and that downstream analytics and reporting activities rest on a solid evidentiary foundation.

The system can integrate a variety of datasets, including medical records, social engagement data, family history, and personal preferences, to generate personalized care plans for each care recipient. By leveraging data from wearables, family surveys, health assessments, and care recipient histories, the system can continuously adapt to meet the evolving needs of each individual.

For example, by integrating medical data from caregiver inputs and health assessments, the system can create personalized care plans based on a care recipient's specific health conditions, cognitive status, and medication requirements. If a care recipient is diagnosed with dementia or heart disease, the system can monitor real-time heart rate variability and steps taken to identify deviations from expected activity levels. For instance, if a care recipient with hypertension shows a consistently elevated average heart rate, the system can recommend adjustments to their medication or dietary intake, or even suggest lower-impact exercises to help regulate their blood pressure.

In addition to health data, the system can also use social engagement data to enhance the care plan. For example, if a care recipient has preferred activities such as gardening, cooking, or watching TV, the system can recommend engaging in these activities to maintain or improve their emotional well-being. If the system detects that a care recipient's social engagement is minimal—indicated by low activity levels or limited interaction with family—it can suggest more frequent family calls or in-person visits, based on the recipient's preferences and family relationships. For instance, if a care recipient is noted to have a preference for watching TV, the system can encourage family members to join in for a shared TV session or suggest family games to further social engagement.

The system can also take into account family history data, such as genetic risks and mental health history, to further personalize care plans. For example, if a care recipient has a family history of heart disease, the system can suggest more frequent cardiovascular check-ups, including monitoring of steps taken to ensure the individual remains active. Similarly, if there is a family history of anxiety or depression, the system can recommend mental health interventions, such as cognitive exercises or stress-reduction techniques, and adjust care plans based on family member inputs regarding mental health concerns.

Real-time behavioral and activity monitoring also plays a critical role. For example, the system can track a care recipient's activity level and steps taken using wearable devices. If the system detects a drop in steps taken, the care plan can be updated to recommend increased physical therapy or even suggest specific exercises to maintain mobility. If a care recipient with arthritis shows decreased mobility, the system can suggest gentle exercises such as range-of-motion activities or stretching to improve joint flexibility.

The integration of family survey responses also allows the system to refine its recommendations. For instance, if a family member indicates that the care recipient “needs someone to motivate them to stay active,” the system can automatically generate activity reminders or suggest specific engagement activities, such as encouraging outdoor walks or participating in virtual games with family members. Additionally, if a family member expresses concern that the care recipient “needs more mental stimulation,” the system can recommend cognitive exercises like puzzles or reading activities to address those needs. These suggestions can be dynamically adjusted based on feedback from caregivers or the care recipient themselves.

As new data is continuously collected, the system can adapt and refine care plans based on the most up-to-date information. For example, if a care recipient begins to show signs of social isolation based on reduced activity levels, the system can suggest increasing interaction with family members or joining social activities aligned with their preferred activities, such as a family gardening session or a shared cooking activity. Similarly, if there is a sudden decline in mobility, the system can trigger an update in the care plan to include additional mobility aids or suggest home modifications like installing grab bars in bathrooms to ensure safety.

In summary, the system can integrate data from a variety of sources to provide a comprehensive, personalized care plan that responds dynamically to the care recipient's changing needs. By incorporating medical data, social engagement metrics, family history, and personal preferences, the system can offer tailored recommendations to improve health outcomes and emotional well-being. These recommendations can be adjusted overtime based on continuous data collection, allowing caregivers to remain informed and proactive in their care delivery, ensuring that each care recipient receives the best possible care.

Directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

In addition, when a component, part or layer is referred to as being “joined with,” “on,” “engaged with,” “adhered to,” “secured to,” or “coupled to” another component, part or layer, it may be directly joined with, on, engaged with, adhered to, secured to, or coupled to the other component, part or layer, or any number of intervening components, parts or layers may be present. In contrast, when an element is referred to as being “directly joined with,” “directly on,” “directly engaged with,” “directly adhered to,” “directly secured to,” or “directly coupled to” another element or layer, there may be no intervening elements or layers present. Other words used to describe the relationship between components, layers and parts should be interpreted in a like manner, such as “adjacent” versus “directly adjacent” and similar words. As used herein, the term “and/or” includes any and all combinations of one or more of the associated listed items.

The above description is that of current embodiments of the invention. Various alterations and changes can be made without departing from the broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, any combination of X, Y and Z, for example, X, Y, Z; X, Y; X, Z; Y, Z, and/or any other possible combination together or alone of those elements, noting that the same is open ended and can include other elements.

Reference throughout this specification to “a current embodiment” or “an embodiment” or “alternative embodiment” means that a particular feature, structure, or characteristic described in connection with the embodiment is included in at least one embodiment herein. Accordingly, the appearance of the phrases “in one embodiment” or “in an embodiment” or “in an alternative embodiment” in various places throughout this specification are not necessarily all referring to the same embodiment. Furthermore, the particular features, structures, or characteristics may be combined in any suitable manner with or in one or more embodiments.

1. A system for authenticating Activities of Daily Living (ADLs) within a residential care environment, comprising:
a plurality of monitoring devices, each disposed proximate to a corresponding zone within the residential care environment and configured to detect one or more forms of activity occurring within that zone, the monitoring devices further configured to generate activity data including at least a zone identifier, a timestamp, and one or more activity-related parameters;
a caregiver device associated with a registered caregiver identifier, the caregiver device configured to interact with one or more monitoring devices to facilitate detection of caregiver-related activity within one or more zones of the residential care environment, and further configured to transmit the caregiver identifier for correlation with activity data generated by the monitoring devices;
a care recipient device associated with a registered care recipient identifier, the care recipient device configured to interact with one or more monitoring devices to facilitate detection of care recipient-related activity within one or more zones of the residential care environment, and further configured to transmit the care recipient identifier for correlation with activity data generated by the monitoring devices;
an ADL event engine configured to:
receive activity data generated by the monitoring devices;
receive the caregiver identifier transmitted by the caregiver device and the care recipient identifier transmitted by the care recipient device;
evaluate the activity data against a plurality of stored ADL event profiles, each profile comprising a set of characteristic features corresponding to a predefined care-related activity, and identify a candidate ADL event when the activity data conforms to one or more of the stored event profiles; and
determine whether the candidate ADL event corresponds to the caregiver identifier, the care recipient identifier, or both, by correlating the caregiver identifier and the care recipient identifier with the activity data based on spatial and temporal alignment,
wherein the caregiver device and care recipient device are each configured to operate in either caregiver or recipient mode, and the system is further configured to assign functional roles based on registered user context or pre-established care schedules, and wherein identity correlation of the caregiver device or the care recipient device is dynamically updated based on detected engagement patterns or presence within a zone during an activity period, and
wherein at least a portion of the activity data is processed locally at a monitoring device or a residential gateway to perform event detection, identity correlation, or activity classification, wherein the transmitted activity data excludes raw visual or audio content and comprises structured activity event data associated with one or more of the registered caregiver identifier and the registered care recipient identifier.

2. The system of claim 1, wherein authentication of the candidate ADL event is contingent upon corroborating activity data from at least two independent sensing modalities, the corroborating activity data each being indicative of the candidate ADL event.

3. The system of claim 1, wherein the spatial alignment is further based on a comparison between the zone identified in the activity data and an expected location associated with the caregiver identifier or the care recipient identifier.

4. The system of claim 1, wherein the activity-related parameters include at least two different sensing modalities selected from: acoustic signals, visual observations, environmental gas concentration levels, appliance state data, and wireless proximity indicators.

5. The system of claim 4, wherein authentication of a candidate ADL event is contingent upon at least two of the sensing modalities independently indicating the same activity condition.

6. (canceled)

7. The system of claim 1, wherein the monitoring devices comprise wearable devices associated with distinct caregiver identifiers, each wearable device configured to monitor caregiver activities within a plurality of zones of the care environment.

8. The system of claim 1, wherein the plurality of stored ADL event profiles includes separate classifications for:
hygiene-related activities,
nutritional intake activities,
mobility-related activities, and
household maintenance activities.

9. The system of claim 8, wherein authentication thresholds associated with stored ADL event profiles are adjustable based on the classified activity type.

10. The system of claim 1, wherein authentication of the candidate ADL event is further conditioned upon detecting sustained activity indicative of the candidate ADL event over a predefined activity duration threshold.

11. The system of claim 10, wherein the activity duration threshold is determined based on the ADL event profile associated with the candidate ADL event.

12. (canceled)

13.-31. (canceled)
